(19) United States
(12) Patent Application Publication
(10) Pub. No.: US 2013/0296417 A1
(43) Pub. Date: Nov. 7, 2013

(54) Theaflavin Compositions, Production, and Method to Control Physiological Disorders in Mammals

(71) Applicants: Xiaowei Shi, Edison, NJ (US); Xueyn Ren, Wuxi (CN); Jiannong Liu, Wuxi (CN); Wenhao Ren, Wuxi (CN); Yiznou Cao, Jiangyin (CN)

(72) Inventors: Xiaowei Shi, Edison, NJ (US); Xueyn Ren, Wuxi (CN); Jiannong Liu, Wuxi (CN); Wenhao Ren, Wuxi (CN); Yiznou Cao, Jiangyin (CN)

(73) Assignee: JANGSU DEHE BIO-TECH CO., LTD., Jiangyin (CN)

(21) Appl. No.: 13/931,674

(22) Filed: Jun. 28, 2013

Related U.S. Application Data
(62) Division of application No. 12/849,996, filed on Aug. 4, 2010.

(51) Int. Cl. A61K 31/353

(52) U.S. Cl. CPC A61K 31/353
USPC 514/456; 435/125

(57) ABSTRACT

A process for producing a purified extract comprising between about 40% and about 90% theaflavins is provided. Purified theaflavin extract produced by the disclosed process is provided which comprises less than about 5% TF1, between about 10% and about 60% TF2a, between about 5% and about 35% TF2b, and between about 10% and about 65% TF3. Individual dosage compositions are provided for the control of a physiological disorder comprising about 5% to about 95% theaflavins in a pharmaceutically acceptable vehicle or a dietary supplement vehicle. Further individual dosage compositions are provided which comprise an effective amount of substantially only one theaflavin species selected from the group consisting of TF1, TF2a, TF2b, and TF3. Individual dosage compositions are provided which comprise an effective amount of substantially only two theaflavin species selected from the group consisting of TF1 and TF2a, TF1 and TF2b, TF1 and TF3, TF2a and TF2b, TF2a and TF3, TF2b and TF3. Methods of treatment of human physiological disorders are provided which comprise administering oral dosage forms of the compositions.

This application is a continuation in part of application Ser. No. 12/574,862 filed Oct. 7, 2009.

### FIELD OF THE INVENTION

The present invention relates to enhanced production of purified modified theaflavin extracts using Camellia sinensis var. assamica, oral dosage forms, individual dosage compositions of TF1, TF2a, TF2b, and TF3, and methods of use of the extracts and compositions for the control and/or treatment of mammalian physiological disorders.

### BACKGROUND OF THE INVENTION

Tea is produced from the leaves of Camellia Sinensis and contain significant amounts of polyphenol compounds. Theaflavins, which comprise a mixture of theaflavin (TF1), theaflavin-3-gallate (TF2A), theaflavin-3′-gallate (TF2B) and theaflavin-3,3′-digallate (TF3), as depicted in FIG. 2, are typically formed via polymerization of green tea polyphenols (catechins) during fermentation of green tea to black tea. Typically, the concentration of theaflavins in black tea is between about 0.4% to about 1.8% by weight with the concentration of theaflavins in green tea usually being far less.

Theaflavin (catechin dimer joined at B rings) and its derivatives, known collectively as theaflavins, are antioxidant polyphenols Flavan-3-ols formed from catechins during enzymatic oxidation (fermentation). Theaflavins generally comprise a mixture of theaflavin (TF1), theaflavin-3-gallate (TF2A), theaflavin-3′-gallate (TF2B) and theaflavin-3,3′-digallate (TF3).

Influenza is a highly contagious, acute respiratory disease that has afflicted humans since ancient times. With influenza viruses as causative agents, this infectious viral illness is a major cause of morbidity and mortality in communities (epidemics) and worldwide (pandemics). Its rapid propagation is normally due to high infectivity of the virus, short incubation period as well as high titer in respiratory secretions during the shedding phase. The primary way of spread of human influenza is by virus-laden respiratory secretions during coughing and sneezing, with direct or indirect contact representing other possible modes of transmission. After virus replication and spread of infection throughout the upper and lower airways, virus is shed in nasopharyngeal secretions for 5 to 10 days. Following an incubation period of 1 to 4 days, a broad spectrum of symptoms also known as “flulike” symptoms, comprise febrile illness accompanied by variable respiratory disease with or without systemic features, including cough, rhinorrhea, headache, malaise and myalgia. Multi-system complications affecting lungs, heart, brain, liver kidney and muscle, as well as death are also possible outcomes due to either primary viral infection or secondary bacterial pneumonia.

The impact of influenza infection is felt globally each year when the disease develops in approximately 20% of world's population. In the United States, influenza infections occur in epidemics each winter, generally between late December and early March. Recent events, including human cases of avian influenza, have heightened awareness of the threat of a pandemic and have spurred efforts to develop plans for its control.

Influenza virus is a member of the Orthomixoviridae family, which is an enveloped virus with a segmented negative-stranded RNA (ssRNA). Three different types of influenza (A, B, and C) can be distinguished by antigenic differences between their nucleocapsid (NP) and matrix (M) proteins. Also, influenza virus type A is further subtyped according to variations in the surface glycoproteins hemagglutinin (HA) and neuraminidase (NA).

Current and prospective antiviral agents form an important part of a rational approach to epidemic influenza and are critical to planning for a pandemic. During any influenza season, antigenic drift in the virus may occur after formulation of the year's vaccine has taken place, rendering the vaccine less protective, and outbreaks can more easily occur among high-risk populations. In the course of a pandemic, vaccine supplies are inadequate. Vaccine production by current methods cannot be carried out with the speed required to halt the progress of a new strain of influenza virus; therefore, it is likely that vaccine would not be available for the first wave of spread of virus.

Theaflavin (catechin dimer joined at B rings) and its derivatives, known collectively as theaflavins, are antioxidant polyphenols Flavan-3-ols formed from catechins during enzymatic oxidation (fermentation). Theaflavins generally comprise a mixture of theaflavin, theaflavin-3-gallate, theaflavin-3′-gallate and theaflavin-3,3′-digallate.

Theaflavins have positive health benefits directly linked to the antioxidant properties of these compounds. The benefits include the ability to effect lower blood lipid levels (e.g. cholesterol), control of inflammation, as well as anti-tumor effects. See, e.g., Maron D J, et al., Cholesterol-Lowering Effect of a Theaflavin-Enriched Green Tea Extract: a Randomized Controlled Trial, Arch. Intern. Med. 163 (12): 1448 (2003). See, also, Lorentz, M., et al., Basic Res Cardiol. 2009 January; 104 (1): 100; Manna, S., et al., J Nutr Biochem. 2009 May; 20 (5):337.

Limited availability of theaflavins, economically derived from natural sources, however, presents a significant barrier to realization of the health benefits of theaflavins. Commercial reagents such as polyphenol peroxidase and hydrogen peroxide from commercial sources are often used in the fermentation step, which further increases the cost of the production. Goodsall, et al., U.S. Pat. Nos. 6,833,144; 6,113,965. Many current extraction and purification steps employ environmentally unfriendly solvents methanol and chloroform. Environmental hazards moreover result from current processes due to the frequent application of strong acids and bases and large amounts of resulting waste generated.

An ongoing need for low cost, high yield, environmentally friendly scalable production of theaflavins from natural sources indeed exists toward realization of numerous health benefits. Furthermore, although vaccination is the primary strategy for the prevention of influenza, for example, there are a number of scenarios for which vaccination is inadequate; and, as a corollary, the quest for effective antiviral agents is of utmost importance.

### SUMMARY OF THE INVENTION

The present invention is directed to an enhanced process for producing a purified extract comprising between about 40% and about 90% theaflavins which comprises combining an organic solvent with tea leaves, extracting catechins (polyphenols) from the tea leaves to produce an organic stock substrate solution; producing a second batch of tea leaves; grinding the second batch of tea leaves to produce stock fermentation enzyme; combining the stock substrate solution with the stock fermentation enzyme to produce a fermentation mixture; fermentation of the mixture to produce theaflavins; and, separating the theaflavins from the fermentation mixture to produce theaflavins. The yield of the purified theaflavin from large tea leaves is unexpectedly about 30-60% higher than conventional extraction processes.

The invention is directed to an alternate process for producing purified theaflavin extract comprising between about 40% and about 90% theaflavins, less than about 5% TF1, between about 10% and about 60% TF2a, between about 5% and about 35% TF2b, and between about 10% and about 65% TF3, which comprises combining an organic solvent with tea leaves, extracting polyphenols from the tea leaves to produce an organic substrate solution; concentrating the organic substrate solution, then washing the organic substrate solution with water to produce an organic stock substrate solution having certain monomers of catechins. This process further comprises producing a second batch of tea leaves; grinding the second batch of tea leaves to produce stock fermentation enzyme; combining the stock substrate solution with the stock fermentation enzyme to produce a fermentation mixture; fermentation of the mixture to produce theaflavins; and, separating the theaflavins from the fermentation mixture and further purifying theaflavins through macroporous absorbent resin to produce the high purity of theaflavin extract with specific proportion of monomers of theaflavins.

The invention is further directed to an oral dosage form comprising an effective amount of a purified extract produced by the process of the invention.

In addition, the current invention is directed to an individual dosage composition for the control of a physiological disorder comprising an effective amount, between about 5% to about 95% of substantially only one theaflavin species selected from the group consisting of TF1, TF2a, TF2b, and TF3, in a pharmaceutically acceptable vehicle or a dietary supplement vehicle.

The current invention is, further directed to an individual dosage composition for the control of a physiological disorder comprising an effective amount, between about 5% to about 95% of substantially only two theaflavin species selected from the group consisting of TF1 and TF2a, TF1 and TF2b, TF1 and TF3, TF2a and TF2b, TF2a and TF3, TF2b and TF3, in a pharmaceutically acceptable vehicle or a dietary supplement vehicle.

The current invention is, further directed to an individual dosage composition for the control of a physiological disorder comprising an effective amount, between about 5% to about 95% of substantially only three theaflavin species selected from the group consisting of TF1 and TF2a and TF2b, TF3 and TF2a and TF2b, TF3 and TF1 and TF2b; and, TF3 and TF1 and TF2a, in a pharmaceutically acceptable vehicle or a dietary supplement vehicle.

The current invention is, further directed to an individual dosage composition for the control of a physiological disorder comprising an effective amount, between about 5% to about 95% of substantially only theaflavin species TF1, TF3, TF2a, and TF2b, in a pharmaceutically acceptable vehicle or a dietary supplement vehicle.

The invention is also directed to methods to control and/or treatment of a mammalian physiological disorder comprising administering an effective amount of an oral dosage form or an individual dosage composition of theaflavin extracts and compositions described herein.

### BRIEF DESCRIPTION OF THE FIGURES

FIG. 1 displays a process embodiment of the present invention for manufacturing theaflavins.

FIG. 2 shows the structures of theaflavins (TF1), theaflavin-3-gallate (TF2A), theaflavin-3′-gallate (TF2B) and theaflavin-3,3′-digallate (TF3).

FIG. 3 shows the structures of the four main polyphenol substrates for enzymatic fermentation to produce theaflavins, e.g., (−)-epigallocatechin-3-gallate (EGCG), (−)-epigallocatechin (EGC), and (−)-epicatechin-3-gallate (ECG), (−)-epicatechin (EC).

FIG. 4 displays an alternative process of the present invention for manufacturing theaflavins.

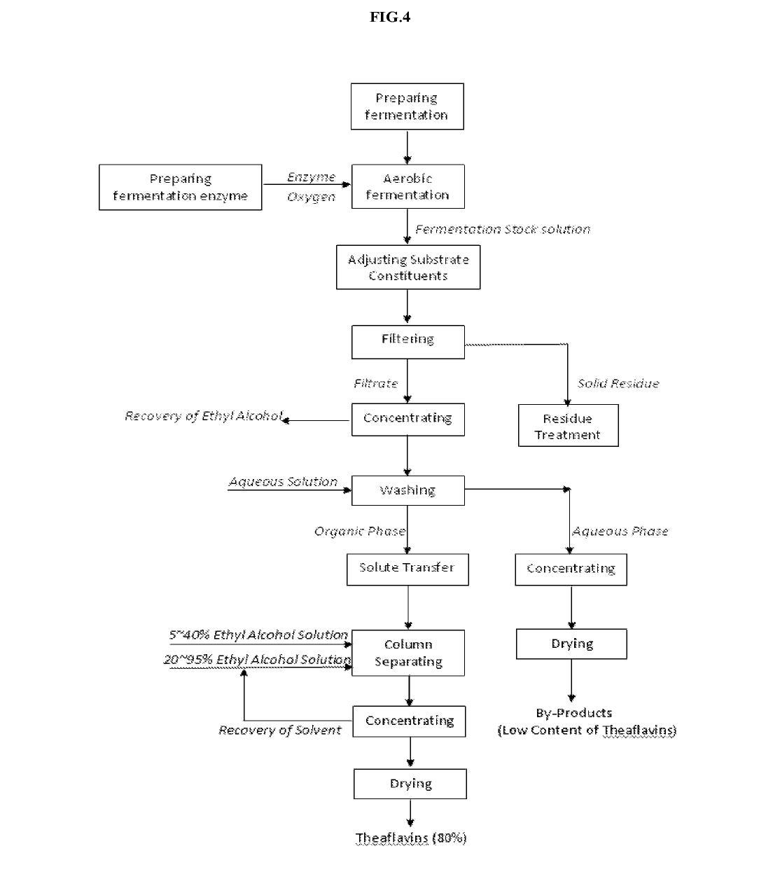

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference including U.S. Pat. Nos. 7,427,622; 7,157,493 and 7,288,680.

The term eluting solvent refers to one or more solvents in various ratio, including but not limited to ethyl acetate, hexane, water, acid in water, acetone, acetonitrile, and methanol.

The term separation medium includes, but is not limited to, silica gel, Sephadex LH20, Toyopearl HW series, C-18 Reverse Phase material, D101 macroporous resin, or a combination of the above.

The term organic stock substrate solution, as used herein, refers to an organic tea leaf extract containing polyphenols.

The term stock fermentation enzyme, as used herein, refers to a source of endogenous enzymes in tea leaves, polyphenol oxidase (PO) and polyphenol peroxidase (PPO) for example.

The term fermentation mixture, as used herein, refers to an organic tea leaf extract containing polyphenols mixed with endogenous enzymes in tea leaves and wherein the polyphenols undergo oxidation to flavins by means of the enzymes including polyphenol oxidase (PO) and polyphenol peroxidase (PPO).

The term water, as used herein has its ordinary meaning, but includes acidic aqueous basic aqueous.

The term human physiological disorder as used herein includes but is not limited to inflammation, influenza infection, hyperlipidemia, coronary heart disease, apoplexy, arthesclerotic cardiovascular diseases, AIDS, diabetes, oxidated-low density lipoprotein level, von Willebrand's disease, leukopenia, cerebral infarction, dementia and physical disorder, and fatty liver.

The term oral dosage form as used herein includes but is not limited to a tablet, capsule, powder, solution, suspension, emulsion, pill, pellet, sustained-release formulation that contain an effective amount of theaflavins produced by methods according to the present invention. However, since theaflavins produced by methods according to the present invention are incorporated into food items such as nutritional bars and nutritional drinks the term oral dosage form includes food and drink items that incorporate an effective amount of theaflavins produced by methods according to the present invention.

Fermentation refers to an oxidative process to produce theaflavins, and optionally thearubigins.

Fermentation enzyme refers to the native reagents of tea leaves which effect fermentation. Preferrably whole tea leaves optionally treated with a solvent, preferably moisturized with pure water or 0.1%-1% of citric acid solution, frozen, and then ground, preferably to particles, for example, smaller than about 80 mesh.

The term “washing” refers to a process of mixing thoroughly an organic phase with an aqueous phase, separating the organic phase from the aqueous phase, and collecting the organic phase. Optionally, this process can be repeated one or more times and the organic phases are combined.

Solute transferring refers to a process of adding an aqueous phase to an organic phase to form a mixture, concentrating said mixture and forcing the solute in the organic phase to be transferred into the aqueous phase.

### Influenza

According to the current nomenclature system, strains of influenza virus are designated with the type (based on the antigenic specificity of the NP antigen, i.e. A, B or C), host of origin (indicated only for non-human hosts), and geographic location of first isolation, strain number, and year of isolation. For influenza A viruses the antigenic description (HA and NA) is provided in parenthesis following the strain designation (e.g. A/PR/8/34 (H1N1), A/HongKong/8/68 (H3N2), A/Swine/Taiwan/70 (H3N2), B/Russia/69, C/California/78) (286).

All three influenza viruses infect humans and cause disease; however, they have different epidemiologic characteristics:
1. Type A viruses are the cause of severe recurrent epidemic diseases with high mortality in humans; they can also infect other mammals such as pigs, horses, seals, as well as a variety of domestic and wild birds (e.g. swine, chickens, turkey, ducks, geese). Influenza A viruses are perpetuated in nature by wild birds, especially shorebirds and waterfowl. Fifteen HA and nine NA subtypes have been identified to date but only three types of HA (H1, H2 and H3) take part in influenza A virus attachment to human cells, and two types of NA (N1 and N2) are in charge of virus penetration.
2. Influenza B viruses display a higher immunologic stability than those from type A. It has been isolated from seals, but most commonly affects humans causing clusters of infection in closed populations.
3. Influenza C virus is capable of infecting humans, dogs, and swine. Cases of human illness are usually subclinical and therefore rarely reported.

Neuraminidase or NA, is a glycoprotein present on the surface of influenza A and B viruses. Its structure has been described as a homotetramer that forms a spike with a head domain protruding from the cell surface, a trans-membrane domain, and a small cytoplasmic tail. Besides constituting a primary target against which neutralizing antibodies are produced, functional viral neuraminidase (NA) acts as a receptor-destroying enzyme (sialidase) cleaving sialic acid from the HA molecules, other NA and oligosaccharides at the cell surface. For this, NA catalyzes the hydrolysis of specific glycosidic linkages (α2, 6 or α2, 3) between sialic acid and its adjacent carbohydrate moiety.

In addition, NA has been shown to play a role in initial viral infection with the removal of sialyl residues on mucins and cilia (decoy receptors for HA). By evading these natural defenses of the respiratory tract, NA grants virions the access to functional receptors on surface membrane of target cells.

Due to the presence of influenza virus HA on the same cell membrane during release of newly formed virions, cleavage of sialic acid residues by means of influenza virus NA becomes necessary in order to prevent the formation of viral aggregates and to allow spreading of virions to other host cells. Currently, two FDA approved antiviral drugs (oseltamivir and zanamivir) act by inhibiting influenza virus NA blocking the release of progeny virions, thereby reducing viral infectivity.

Oseltamivir is available as a capsule or for liquid suspension with good oral bioavailability. It is readily absorbed from the gastrointestinal tract, is converted by hepatic esterases to the active form of the compound (oseltamivir carboxylate), and is widely distributed in the body. The half-life is 6 to 10 hours. The drug is excreted primarily through the kidneys; thus, dosing must be modified in patients with renal insufficiency. Oseltamivir achieves high plasma levels and thus can act outside the respiratory tract.

All influenza viruses bear two surface glycoproteins, a hemagglutinin and a neuraminidase, which are the antigens that define the particular strain of influenza. The variation of these molecules over time permits the virus to evade human immune responses and therefore necessitates the formulation of a new vaccine each year. The hemagglutinin is a sialic acid receptor-binding molecule and mediates entry of the virus into the target cell. The neruaminidase—the target molecule of the neuraminidase inhibitor compounds—cleaves the cellular-receptor sialic acid residues to which the newly formed particles are attached. This cleavage releases the viruses, which can now invade new cells. Without neuraminidase, infection would be limited to one round of replication, rarely enough to cause disease. Neuraminidase may also facilitate viral invasion of the upper airways, possibly by cleaving the sialic acid moieties on the mucin that bathes the airway epithelial cells.

The ability of transition-state analogues of sialic acid to inhibit the influenza neuraminidase was first recognized in the 1970s, but the design of highly effective inhibitors became feasible when analysis of the three-dimensional structure of influenza neuramimidase disclosed the location and structure of the catalytic site. Potent inhibitors such as zanamivir closely mimic the natural substrate, fitting into the active site pocket and engaging the protein in the most energetically favorable interaction. Zanamivir is administered by oral inhalation, which delivers the drug directly to the respiratory tract. Oseltamivir was developed through modifications to the sialic acid analogue framework (including the addition of a lipophilic side chain) that allow the drug to be used orally.

Given the need for additional antiviral agents as well as their availability and surge capacity, studies described herein were intended to identify new compounds with potential anti-influenza activity. This was done by screening theaflavins (TF) by means of a cell-based system that assayed the inhibition of virus-induced cell death. This HTS assay was developed under stringent standards for high sensitivity, reproducibility, and accuracy. As a result, a group of primary hits was identified that fulfilled the conditions established of cellular protection from virus cytopathic effects with low or no inherent cytotoxicity at the concentration tested. A second screening of this group of compounds narrowed down the number of hits by eliminating false-positives, typical of large high-throughput screens, and confirming those with antiviral activity. More thorough evaluation of the primary hits was done by determination of their 50% inhibitory (IC₅₀) and 50% cytotoxicity concentrations (CC₅₀). Analysis and comparison of these values allowed for the selection “lead” compounds, i.e., those with the most promising therapeutic index (TI).

### Theaflavin Compositions

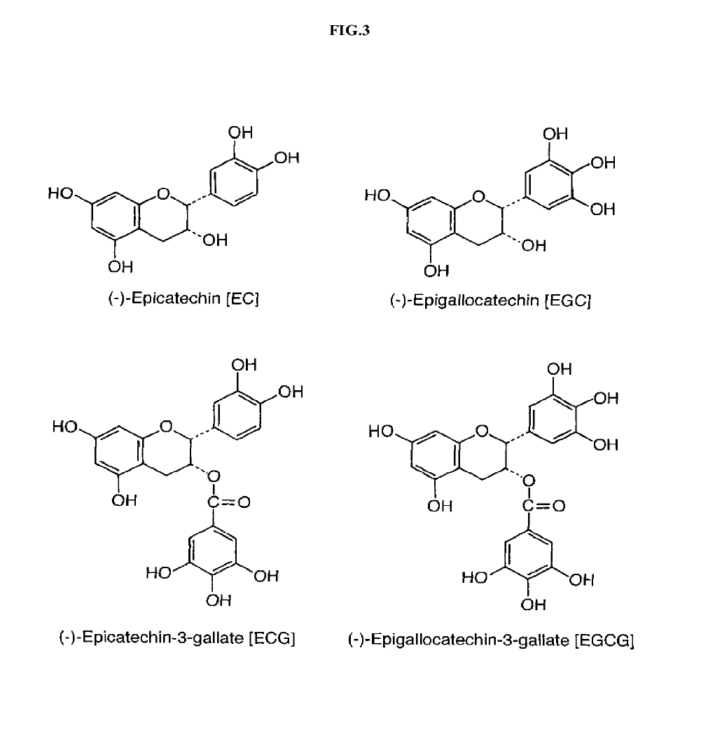

The term “catechins” and “monomers of catechins” generally includes the four main polyphenol substrates for enzymatic fermentation to produce theaflavins, e.g., (−)-epigallocatechin-3-gallate (EGCG), (−)-epigallocatechin (EGC), (−)-epicatechin-3-gallate (ECG), (−)-epicatechin (EC) as well as (+)-gallocatechin, and (+)-catechin. See, FIG. 3.

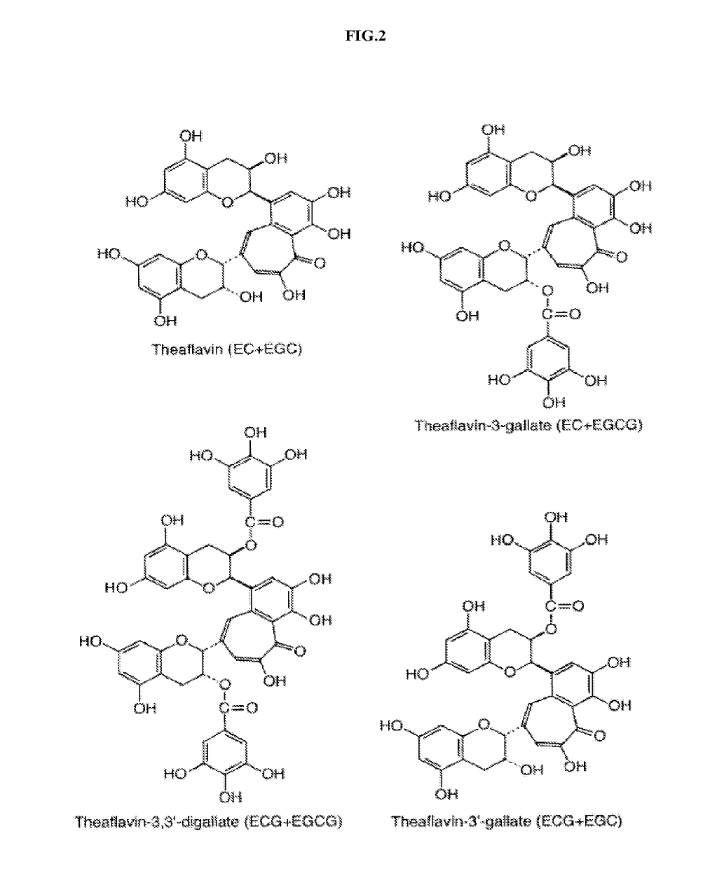

Theaflavin (catechin dimer joined at B rings) and its derivatives, known collectively as theaflavins, are antioxidant polyphenols Flavan-3-ols formed from catechins during enzymatic oxidation (fermentation). Theaflavins generally comprise a mixture of theaflavin (TF1), theaflavin-3-gallate (TF2A), theaflavin-3′-gallate (TF2B) and theaflavin-3,3′-digallate (TF3). See, FIG. 2. As used herein the term theaflavins includes but is not limited to theaflavin, isotheaflavin, neotheaflavin, theaflavin-3-gallate, theaflavin-3′-gallate, theaflavin-3,3′-digallate, epitheaflavic acid, epitheaflavic acid-3′-gallate, theaflavic acid, theaflavic acid-3′-gallate and mixtures thereof. The term includes salt forms of these compounds.

EC, ECG, EGC, and EGCG are major polyphenols for enzymatic oxidation and the production of theaflavins. The overall content of polyphenols is as much as 90% higher in large leaf tea than in small leaf tea. An important feature for large leaf tea is the high percentage of ECG (see Table I), which is a key substrate for the production of TF2b and TF3.

Green tea polyphenols contain about 12 catechins, including EGCG, EGC, ECG, EC.

The rough ratios of the four catechins:
EGCG 50%
EGC 20%
ECG 20%
EC 10%

TABLE A
major polyphenols (catechins) and their relative percentages in tea leaves

| | EC | ECG | EGC | EGCG |
|---|---|---|---|---|
| Small leaf | 10% | 10-20% | 20-50% | 35-50% |
| green tea^{A} | | | | |
| Large leaf | 15-25% | 35-40% | 10% | 30-35% |
| green tea | | | | |

A: Proc Natl Sci Counc Repub China B. 1993 Apr; 17(2): 77-84.

Theaflavins are Currently Produced from Green Tea Leaves to Yield Purified Compositions Wherein

TF1 is generally within the range of 40-50%, TF2A is generally within the range of 15 to 25%, TF2B is generally within the range of 10-14% and TF3 is generally within the range of 15-25%.

TABLE B
Components of Theaflavins and their Relative Percentage

| | TF1 | TF2a | TF2b | TF3 |
|---|---|---|---|---|
| Polyphenol | EC + EGC | EC + EGCG | ECG + EGC | ECG + EGCG |
| substrate | | | | |
| From regular | 40-50% | 15-25% | 10-14% | 15-25% |
| green tea | | | | |
| From large | ≦4 | 20-50% | 5-20% | 20-50% |
| leaf tea | | | | |

Fermented Tea contains theaflavins, including, TF1, TF2A, TF2B, TF3.

The rough ratios of the four theaflavins:
TF1: 5%
TF2A: 25%
TF2B: 10%
TF3: 60%

### Large Leaved Tea Confer Advantage as to Production and Theaflavin Compositions

Comparing with the small-leaf species, Yunnan large-leaf tea has about 30-60% higher content of catechins, which are polyphenols and undergo oxidation to produce theaflavins.

Certain process embodiments for production of theaflavins described herein employs large tea leaves, preferrably leaves from Yunnan large leaf tea, as a source of catechins. As a result, the yield of theaflavins produced by this process is improved by about 30-60% compared with processes using small tea leaves.

The botanical name of tea is Camellia sinensis which has two principal varieties: Camellia sinensis var. sinensis and Camellia sinensis var. assamica. Large leaf tea belongs to the variety of Camellia sinensis var. assamica. Yunnan large-leaf tea, classified into Camellia sinensis var. assamica, include Mengku, Fengqing, Menghai, and Jingdong.

Large leaved tea exhibits strong carbon metabolism so that polyphenols, total catechins, catechin esters (ECG) and ratio of phenols/ammonia are high comparing to small leaved tea.

It is an ideal candidate for producing theaflavins, black tea and Puer tea. Contrasting, small leaved tea shows strong metabolism of ammonia and amino acids that present high contents of ammonia, amino acids and theines. L-EGCG is normally highest content, and then following L-ECG and L-EGC in catechins presented in tea. However, L-ECG content is almost same as L-EGCG in Yunnan large leaved tea, and total content of L-EC, D-Catechin and L-Catechin is closed to L-EGC. In large leaved tea, ester type of catechins is higher than that in small leaved tea, which indicates the potential higher yield of theaflavins during process of oxidation by polyphenol oxidase.

### (1) Morphological Character

Camellia sinensis L. or the China tea plant is a big shrub, 1-2 m tall with many virgate stems arising from the base of the plant near the ground. Leaf hard, thick and leathery; surface matt, marginal veins indistinct and appear sunken in lamina. Blade elliptic with obtuse or broadly obtuse apex; base cuneate, margin bluntly serrulate to sinuate-serrulate with more or less incurved teeth, glabrous above and villose below when young, becoming sparsely villose as the leaf ages. Ultimately becoming glabrous. Young leaves garnet-brown through α-blood to purple in colour. Petiole short, 3-7 mm long, stout, usually giving the leaf an erect pose.

Camellia assamica (Masters) or the Assam tea plant is a tree. 10-15 m tall with a trunk sometimes up to one third of its height, possesses a robust branch system. In typical plants, leaf dependent, thin, and glossy with more or less acuminate apex and distinct marginal veins. Leaf blade usually broadly elliptic, 8-20 cm long and 3.5-7.5 cm wide, base cuneate, margin obscurely denticulate to bluntly wide-serrulate, glabrous or persistently hairy on the midrib below. The thickness of horny layer in large leaved tea is about 2-4 μm (small leaved tea: 4-8 μm); the palisade tissue in large leaved tea normally has 1 layer (small leaved tea is normally up to 2-3 layers); the amount of chloroplast in the cell of palisade tissue is higher than that of small leaved tea that indicates its photosynthetic rate is relatively high and active so that the large leaved tea shows yellow-green color instead of dense and green color like small leaved tea; the spongy parenchyma flourishes in large leaved tea and the back pore stoma is large and thin that indicate the synthesis of polyphenols and theine are more active; and less pore stoma per unit blade area means more stomata guard cell in large leaved tea, which it has high rate of transpiration.

Large leaved tea exhibit higher content of polyphenols, catechins and theines than those of small leaved tea. Small leaved tea with more palisade tissue where is a synthetical location for xanthin and carotenes, etc. that are the major contributors for the characteristic aromatics and flavors of the tea, is more suitable for brewing tea and instant tea. Table C shows a brief comparisons between large and small leaved tea.

TABLE C
Comparisons of Characteristics of Large Leaf and Small Leaf Teas

| Characteristics | Large Leaf | Small Leaf |
|---|---|---|
| Thickness of horny layer of | 2-4 μm | 4-8 μm |
| the leaf | | |
| Palisade tissue of the leaf | 1 layer, normally | 2-3 layers |
| Chloroplast in Palisade tissue | 60-100 lamina | 20-40 |
| Xanthin | Low | High |
| Carotene | Low | High |
| photosynthetic rate | High | Low |
| Spongy parenchyma | More | Less |
| Polyphenols | High | Low |
| Theine | High | Low |
| Ration of Palisade tissue/ | 1:2-1:3 | 1:1-1:1.5 |
| Spongy parenchyma | | |
| Pore stoma in back of leaf | Large and thin | More and dense |
| Number of stoma in unit | Less | more |
| blade area | | |
| Stomata guard cell | Large | Small |
| Stomata frequency | 100/mm²-180/mm² | 200/mm²-260 mm² |
| transpiration rate | Fast | Slow |

### (2) Principal Chemical Constituents Presented in Large Leaf Tea and Small Leaf Tea Table D Shows Comparisons of Principal Chemical Constituents in Large Leaved and Small Leaved Tea.

TABLE D
Comparisons of Chemical Constituents in Different Tea Varieties (%)

| | Water | | | Amino | Phenols/ |
|---|---|---|---|---|---|
| Variety | Extract | Caffeine | Polyphenols | Acid | ammonia |
| Large Leaved | 45.3 | 4.44 | 38.7 | 1.88 | 20.59 |
| Tea 1 | | | | | |
| Large Leaved | 49.9 | 4.1 | 42.3 | 1.8 | 23.5 |
| Tea 2 | | | | | |
| Small Leaved | 46.8 | 3.74 | 21.62 | 4.59 | 4.7 |
| Tea 1 | | | | | |
| Small Leaved | — | — | 20.85 | 3.97 | 5.2 |
| Tea 2 | | | | | |

1—Yunnan Large Leaved Tea
2—Yun Kang N0. 10
3—Fu Ding White Tea
4—Long Jing Tea

### Purified Theaflavin Extracts (Amount of Theaflavins)

The term “about” as used herein generally includes a 10% margin on either side of the set value (so about 3%, for example, includes 2.7%-3.3%).

Example solid purified extract compositions produced by the processes described herein contain between about 50% and 90% theaflavins. Alternate exemplary compositions produced by the processes described herein contain between about 60% and 85% theaflavins. Further compositions produced by the processes described herein contain between about 70% and 80% theaflavins. Exemplary compositions produced by the processes described herein generally contain about 80% theaflavins. These solid purified extract compositions of the present invention contain less than about 5% TF1, between about 20 and about 50% TF2a, between about 5 and about 15% TF2b, and between about 20 and about 50% TF3. Alternate solid purified extract compositions of the present invention include less than about 4% TF1, between about 25 and about 40% TF2a, between about 5 and 15% TF2b, and between about 25 and about 40% TF3. Further compositions include less than about 3% TF1, between about 25 and about 35% TF2a, between about 8 and 12% TF2b, and between about 25 and about 35% TF3. Purified solid extract compositions of the present invention generally comprise about 80% theaflavins and include less than about 3% TF1, about 30% TF2a, about 10% TF2b, and about 40% TF3.

A DMSO solution, for example, of theaflavins produced by the method described herein was prepared containing less than about 3% TF1, about 30% TF2a, about 10% TF2b, and about 40% TF3. This exemplary composition was screened as described infra against H1N1 influenza virus. The exhibited rate of inhibition was greater than 65%, and the 50% inhibitory concentration (IC₅₀) is 30 μg/mL. At cellular level, the rate of inhibition for H1N1 virus is 80% with an IC₅₀ of 8.7 μg/mL. The 50% cytotoxicity concentration (CC₅₀) is greater than 100 μg/mL. The therapeutic index (CC₅₀/IC₅₀) is greater than 11.5.

### TF1, TF2a, TF2b, and TF3 Individually

Theaflavins can be purified by means including, but not limited to filtration, recrystallization, and chromatography. Theaflavin compositions containing TF1, TF2a, TF2b, and TF3 are dissolved in a suitable solvent, for example 40% ethyl acetate/hexane, and then loaded onto a column filled with a separation medium, for example silica gel. The column is eluted with an organic solvent or a mixture of organic solvents, for example 40% ethyl acetate/hexane. A gradient elution can be used if necessary. A UV detector, for example, a Waters 486 UV/VIS detector, is used for monitoring the purification process. Fractions are collected to obtain TF1, TF2a, TF2b, or TF3 individually. The purity and molecular weight of TF1, TF2a, TF2b, and TF3 can be determined by HPLC-MS. Each of these compounds can be further purified by recrystallization from ethanol or by resubmitting them to column purification. Selection of the parameters needed to resolve the mixture of theaflavins into TF1, TF2a, TF2b, and TF3 (i.e., selection of type and amount of chromatographic media, choice of eluting solvent, length of column, etc.) is well within the capability of those of skilled in the art.

### Commercial Source for Theaflavins

Wako Pure Chemical Industries, Ltd. Japan

Theaflavin (TF1), Cat#: 201-15161

Theaflavin-3-gallate (TF2a), Cat#: 202-15191

Theaflavin-3′-gallate (TF2b), Cat#: 204-15271

Theaflavin-3,3′-digallate (TF3), Cat#: 208-15171

### Section for Individual Dosage Compositions of TF1, TF2a, TF2b, and TF3

The following individual dosage compositions of TF1, TF2a, TF2b, and TF3 refer to substantially all (total amount) the theaflavins in the composition dosage form. Preferred compositions described herein comprise theaflavins in a pharmaceutically acceptable vehicle or a dietary supplement vehicle.

Example composition dosage forms described herein this section for individual dosage compositions include dosage forms containing substantially up to about 100% theaflavins. Preferred dosage form embodiments of the present invention comprise about 5% to about 95% theaflavins in a pharmaceutically acceptable vehicle or a diet supplement vehicle. Similarly dosage form embodiments of the present invention comprise about 10% to about 90% theaflavins in a pharmaceutically acceptable vehicle or a diet supplement vehicle. Dosage form embodiments of the present invention comprise about 15% to about 85% theaflavins in a pharmaceutically acceptable vehicle or a diet supplement vehicle. Dosage form embodiments of the present invention comprise about 20% to about 80% theaflavins in a pharmaceutically acceptable vehicle or a diet supplement vehicle. Preferred dosage form embodiments of the present invention comprise about 25% to about 75% theaflavins in a pharmaceutically acceptable vehicle or a diet supplement vehicle. Dosage form embodiments of the present invention comprise about 30% to about 70% theaflavins in a pharmaceutically acceptable vehicle or a diet supplement vehicle. Dosage form embodiments of the present invention comprise about 35% to about 65% theaflavins in a pharmaceutically acceptable vehicle or a diet supplement vehicle. Dosage form embodiments of the present invention comprise about 40% to about 60% theaflavins in a pharmaceutically acceptable vehicle or a diet supplement vehicle. Dosage form embodiments of the present invention comprise about 40% to about 60% theaflavins in a pharmaceutically acceptable vehicle or a diet supplement vehicle. These compositions described herein comprise theaflavins in a pharmaceutically acceptable vehicle or a diet supplement vehicle, in the following proportions I-XIII:

I. Substantially only TF1
II. Substantially only TF2a
III. Substantially only TF2b
IV. Substantially only TF3

### V. Substantially Only TF1 and TF2a

About 5% TF1 (in this case, “Substantially only TF1 and TF2a” the applicants point out for clarity, “about 5% TF1” necessarily requires about 95% TF2a), about 3% TF1 (necessarily requires about 97% TF2a, and so forth in all percent ratios of Individual Dosage Compositions of this section), about 2% TF1, about 1% TF1, about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80% TF1, about 85% TF1, about 90% TF1, about 95% TF1.

VI. Substantially only TF1 and TF2b

About 5% TF1 (in this case, “Substantially only TF1 and TF2b” the applicants similarly point out again, for clarity, “about 5% TF1” necessarily requires about 95% TF2b and so forth in all percent ratios of Individual Dosage Compositions of this section), about 3% TF1, about 2% TF1, about 1% TF1, about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80% TF1, about 85% TF1, about 90% TF1, about 95% TF1.

VII. Substantially only TF1 and TF3

About 5% TF1, about 3% TF1, about 2% TF1, about 1% TF1, about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80% TF1, about 85% TF1, about 90% TF1, about 95% TF1.

VIII. Substantially only TF2a and TF2b

About 5% TF2a, about 3% TF2a, about 2% TF2a, about 1% TF2a, about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a, about 85% TF2a, about 90% TF2a, about 95% TF2a.

VIII. Substantially only TF2a and TF3

About 5% TF2a, about 3% TF2a, about 2% TF2a, about 1% TF2a, about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a, about 85% TF2a, about 90% TF2a, about 95% TF2a.

VIII. Substantially only TF2b and TF3

About 5% TF2b, about 3% TF2b, about 2% TF2b, about 1% TF2b, about 10% TF2b, about 15% TF2b, about 20% TF2b, about 25% TF2b, about 30% TF2b, about 35% TF2b, about 40% TF2b, about 45% TF2b, about 50% TF2b, about 55% TF2b, about 60% TF2b, about 65% TF2b, about 70% TF2b, about 75% TF2b, about 80% TF2b, about 85% TF2b, about 90% TF2b, about 95% TF2b.

IX. Substantially only TF1, TF2a, and TF2b

A. About 5% TF1, the remainder being Substantially only TF2a and TF2b:

About 5% TF2a (the applicants point out for clarity, “about 5% TF2a” necessarily requires about 90% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a, about 85% TF2a, about 90% TF2a.

B. About 10% TF1, the remainder being Substantially only TF2a and TF2b:

About 5% TF2a (the applicants point out for clarity, “about 5% TF2a” necessarily requires about 85% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a, about 85% TF2a, about 85% TF2a.

C. About 15% TF1, the remainder being Substantially only TF2a and TF2b:

About 5% TF2a (the applicants point out for clarity, “about 5% TF2a” necessarily requires about 80% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80%.

D. About 20% TF1, the remainder being Substantially only TF2a and TF2b:

About 5% TF2a (the applicants point out for clarity, “about 5% TF2a” necessarily requires about 75% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a.

E. About 25% TF1, the remainder being Substantially only TF2a and TF2b:

About 5% TF2a (the applicants point out for clarity, “about 5% TF2a” necessarily requires about 70% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a.

F. About 30% TF1, the remainder being Substantially only TF2a and TF2b:

About 5% TF2a (the applicants point out for clarity, “about 5% TF2a” necessarily requires about 65% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a.

G. About 35% TF1, the remainder being Substantially only TF2a and TF2b:

About 5% TF2a (the applicants point out for clarity, “about 5% TF2a” necessarily requires about 60% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a.

H. About 40% TF1, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

I. About 45% TF1, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

J. About 50% TF1, the remainder being Substantially only TF2a and TF2b:

About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

K. About 55% TF1, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

L. About 60% TF1, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

M. About 65% TF1, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

N. About 70% TF1, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

O. About 75% TF1, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

P. About 80% TF1, the remainder being Substantially only TF2a and TF2b:

About 5% TF2a (the applicants point out for clarity, “about 5% TF2a” necessarily requires about 15% TF2b), about 10% TF2a, about 15% TF2a.

Q. About 85% TF1, the remainder being Substantially only TF2a and TF2b:

About 5% TF2a (the applicants point out for clarity, “about 5% TF2a” necessarily requires about 10% TF2b), about 10% TF2a.

R. About 90% TF1, the remainder being Substantially only TF2a and TF2b:

About 5% TF2a (the applicants point out for clarity, “about 5% TF2a” necessarily requires about 5% TF2b).

X. Substantially only TF3, TF2a, and TF2b

A. About 5% TF3, the remainder being Substantially only TF2a and TF2b:

About 5% TF2a (the applicants point out for clarity, “about 5% TF2a” necessarily requires about 90% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a, about 85% TF2a, about 90% TF2a.

B. About 10% TF3, the remainder being Substantially only TF2a and TF2b:

About 5% TF2a (the applicants point out for clarity, “about 5% TF2a” necessarily requires about 85% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a, about 85% TF2a, about 85% TF2a.

C. About 15% TF3, the remainder being Substantially only TF2a and TF2b:

About 5% TF2a (the applicants point out for clarity, “about 5% TF2a” necessarily requires about 80% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80%.

D. About 20% TF3, the remainder being Substantially only TF2a and TF2b:

About 5% TF2a (the applicants point out for clarity, “about 5% TF2a” necessarily requires about 75% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a.

E. About 25% TF3, the remainder being Substantially only TF2a and TF2b:

About 5% TF2a (the applicants point out for clarity, “about 5% TF2a” necessarily requires about 70% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a.

F. About 30% TF3, the remainder being Substantially only TF2a and TF2b:

About 5% TF2a (the applicants point out for clarity, “about 5% TF2a” necessarily requires about 65% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a.

G. About 35% TF3, the remainder being Substantially only TF2a and TF2b:

About 5% TF2a (the applicants point out for clarity, “about 5% TF2a” necessarily requires about 60% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a.

H. About 40% TF3, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

I. About 45% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

J. About 50% TF3, the remainder being Substantially only TF2a and TF2b: About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

K. About 55% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

L. About 60% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

M. About 65% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

N. About 70% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

O. About 75% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

P. About 80% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

Q. About 85% TF3, the remainder being Substantially only TF2a and TF2b:

About 5% TF2a (the applicants point out for clarity, “about 5% TF2a” necessarily requires about 10% TF2b), about 10% TF2a.

R. About 90% TF3, the remainder being Substantially only TF2a and TF2b: About 5% TF2a (the applicants point out for clarity, “about 5% TF2a” necessarily requires about 5% TF2b).

XI. Substantially only TF3, TF1, and TF2b

A. About 5% TF3, the remainder being Substantially only TF1 and TF2b:

About 5% TF1 (the applicants point out for clarity, “about 5% TF1” necessarily requires about 90% TF2b), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80% TF1, about 85% TF1, about 90% TF1.

B. About 10% TF3, the remainder being Substantially only TF1 and TF2b:

About 5% TF1 (the applicants point out for clarity, “about 5% TF1” necessarily requires about 85% TF2b), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80% TF1, about 85% TF1, about 85% TF1.

C. About 15% TF3, the remainder being Substantially only TF1 and TF2b:

About 5% TF1 (the applicants point out for clarity, “about 5% TF1” necessarily requires about 80% TF2b), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80%.

D. About 20% TF3, the remainder being Substantially only TF1 and TF2b:

About 5% TF1 (the applicants point out for clarity, “about 5% TF1” necessarily requires about 75% TF2b), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1.

E. About 25% TF3, the remainder being Substantially only TF1 and TF2b:

About 5% TF1 (the applicants point out for clarity, “about 5% TF1” necessarily requires about 70% TF2b), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1.

F. About 30% TF3, the remainder being Substantially only TF1 and TF2b:

About 5% TF1 (the applicants point out for clarity, “about 5% TF1” necessarily requires about 65% TF2b), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1.

G. About 35% TF3, the remainder being Substantially only TF1 and TF2b:

About 5% TF1 (the applicants point out for clarity, “about 5% TF1” necessarily requires about 60% TF2b), about 10% TF1, about 15% TF1, about 20% TF1, about 25% TF1, about 30% TF1, about 35% TF1, about 40% TF1, about 45% TF1, about 50% TF1, about 55% TF1, about 60% TF1.

H. About 40% TF3, the remainder being Substantially only TF1 and TF2b:

About 5% TF1 (the applicants point out for clarity, “about 5% TF1” necessarily requires about 55% TF2b), about 10% TF1, about 15% TF1, about 20% TF1, about 25% TF1, about 30% TF1, about 35% TF1, about 40% TF1, about 45% TF1, about 50% TF1, about 55% TF1.

I. About 45% TF3, the remainder being Substantially only TF1 and TF2b:

About 5% TF1 (the applicants point out for clarity, “about 5% TF1” necessarily requires about 50% TF2b), about 10% TF1, about 15% TF1, about 20% TF1, about 25% TF1, about 30% TF1, about 35% TF1, about 40% TF1, about 45% TF1, about 50% TF1.

J. About 50% TF3, the remainder being Substantially only TF1 and TF2b:

About 5% TF1 (the applicants point out for clarity, “about 5% TF1” necessarily requires about 45% TF2b), about 10% TF1, about 15% TF1, about 20% TF1, about 25% TF1, about 30% TF1, about 35% TF1, about 40% TF1, about 45% TF1.

K. About 55% TF3, the remainder being Substantially only TF1 and TF2b:

About 5% TF1 (the applicants point out for clarity, “about 5% TF1” necessarily requires about 40% TF2b), about 10% TF1, about 15% TF1, about 20% TF1, about 25% TF1, about 30% TF1, about 35% TF1, about 40% TF1.

L. About 60% TF3, the remainder being Substantially only TF1 and TF2b: About 5% TF1 (the applicants point out for clarity, “about 5% TF1” necessarily requires about 35% TF2b), about 10% TF1, about 15% TF1, about 20% TF1, about 25% TF1, about 30% TF1, about 35% TF1.

M. About 65% TF3, the remainder being Substantially only TF1 and TF2b:

About 5% TF1 (the applicants point out for clarity, “about 5% TF1” necessarily requires about 30% TF2b), about 10% TF1, about 15% TF1, about 20% TF1, about 25% TF1, about 30% TF1.

N. About 70% TF3, the remainder being Substantially only TF1 and TF2b:

About 5% TF1 (the applicants point out for clarity, “about 5% TF1” necessarily requires about 25% TF2b), about 10% TF1, about 15% TF1, about 20% TF1, about 25% TF1.

O. About 75% TF3, the remainder being Substantially only TF1 and TF2b:

About 5% TF1 (the applicants point out for clarity, “about 5% TF1” necessarily requires about 20% TF2b), about 10% TF1, about 15% TF1, about 20% TF1.

P. About 80% TF3, the remainder being Substantially only TF1 and TF2b:

About 5% TF1 (the applicants point out for clarity, “about 5% TF1” necessarily requires about 15% TF2b), about 10% TF1, about 15% TF1.

Q. About 85% TF3, the remainder being Substantially only TF1 and TF2b:

About 5% TF1 (the applicants point out for clarity, “about 5% TF1” necessarily requires about 10% TF2b), about 10% TF1.

R. About 90% TF3, the remainder being Substantially only TF1 and TF2b:

About 5% TF1 (the applicants point out for clarity, “about 5% TF1” necessarily requires about 5% TF2b).

XII. Substantially only TF3, TF1, and TF2a

A. About 5% TF3, the remainder being Substantially only TF1 and TF2a:

About 5% TF1 (the applicants point out for clarity, “about 5% TF1” necessarily requires about 90% TF2a), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80% TF1, about 85% TF1, about 90% TF1.

B. About 10% TF3, the remainder being Substantially only TF1 and TF2a:

About 5% TF1 (the applicants point out for clarity, “about 5% TF1” necessarily requires about 85% TF2a), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80% TF1, about 85% TF1, about 85% TF1.

C. About 15% TF3, the remainder being Substantially only TF1 and TF2a:

About 5% TF1 (the applicants point out for clarity, “about 5% TF1” necessarily requires about 80% TF2a), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1, about 80%.

D. About 20% TF3, the remainder being Substantially only TF1 and TF2a:

About 5% TF1 (the applicants point out for clarity, “about 5% TF1” necessarily requires about 75% TF2a), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1, about 75% TF1.

E. About 25% TF3, the remainder being Substantially only TF1 and TF2a:

About 5% TF1 (the applicants point out for clarity, “about 5% TF1” necessarily requires about 70% TF2a), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1, about 70% TF1.

F. About 30% TF3, the remainder being Substantially only TF1 and TF2a:

About 5% TF1 (the applicants point out for clarity, “about 5% TF1” necessarily requires about 65% TF2a), about 10% TF1, about 15% TF1, about 20% TF1, about 25% TF1, about 30% TF1, about 35% TF1, about 40% TF1, about 45% TF1, about 50% TF1, about 55% TF1, about 60% TF1, about 65% TF1.

G. About 35% TF3, the remainder being Substantially only TF1 and TF2a:

About 5% TF1 (the applicants point out for clarity, “about 5% TF1” necessarily requires about 60% TF2a), about 10% TF1, about 15% TF1, about 20% TF1, about 25% TF1, about 30% TF1, about 35% TF1, about 40% TF1, about 45% TF1, about 50% TF1, about 55% TF1, about 60% TF1.

H. About 40% TF3, the remainder being Substantially only TF1 and TF2a:

About 5% TF1 (the applicants point out for clarity, “about 5% TF1” necessarily requires about 55% TF2a), about 10% TF1, about 15% TF1, about 20% TF1, about 25% TF1, about 30% TF1, about 35% TF1, about 40% TF1, about 45% TF1, about 50% TF1, about 55% TF1.

I. About 45% TF3, the remainder being Substantially only TF1 and TF2a:

About 5% TF1 (the applicants point out for clarity, “about 5% TF1” necessarily requires about 50% TF2a), about 10% TF1, about 15% TF1, about 20% TF1, about 25% TF1, about 30% TF1, about 35% TF1, about 40% TF1, about 45% TF1, about 50% TF1.

J. About 50% TF3, the remainder being Substantially only TF1 and TF2a:

About 5% TF1 (the applicants point out for clarity, “about 5% TF1” necessarily requires about 45% TF2a), about 10% TF1, about 15% TF1, about 20% TF1, about 25% TF1, about 30% TF1, about 35% TF1, about 40% TF1, about 45% TF1.

K. About 55% TF3, the remainder being Substantially only TF1 and TF2a:

About 5% TF1 (the applicants point out for clarity, “about 5% TF1” necessarily requires about 40% TF2a), about 10% TF1, about 15% TF1, about 20% TF1, about 25% TF1, about 30% TF1, about 35% TF1, about 40% TF1.

L. About 60% TF3, the remainder being Substantially only TF1 and TF2a:

About 5% TF1 (the applicants point out for clarity, “about 5% TF1” necessarily requires about 35% TF2a), about 10% TF1, about 15% TF1, about 20% TF1, about 25% TF1, about 30% TF1, about 35% TF1.

M. About 65% TF3, the remainder being Substantially only TF1 and TF2a:

About 5% TF1 (the applicants point out for clarity, “about 5% TF1” necessarily requires about 30% TF2a), about 10% TF1, about 15% TF1, about 20% TF1, about 25% TF1, about 30% TF1.

N. About 70% TF3, the remainder being Substantially only TF1 and TF2a:

About 5% TF1 (the applicants point out for clarity, “about 5% TF1” necessarily requires about 25% TF2a), about 10% TF1, about 15% TF1, about 20% TF1, about 25% TF1.

O. About 75% TF3, the remainder being Substantially only TF1 and TF2a:

About 5% TF1 (the applicants point out for clarity, “about 5% TF1” necessarily requires about 20% TF2a), about 10% TF1, about 15% TF1, about 20% TF1.

P. About 80% TF3, the remainder being Substantially only TF1 and TF2a:

About 5% TF1 (the applicants point out for clarity, “about 5% TF1” necessarily requires about 15% TF2a), about 10% TF1, about 15% TF1.

Q. About 85% TF3, the remainder being Substantially only TF1 and TF2a:

About 5% TF1 (the applicants point out for clarity, “about 5% TF1” necessarily requires about 10% TF2a), about 10% TF1.

R. About 90% TF3, the remainder being Substantially only TF1 and TF2a:

About 5% TF1 (the applicants point out for clarity, “about 5% TF1” necessarily requires about 5% TF2a).

XIII. Substantially only TF1, TF3, TF2a, and TF2b

A. About 5% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 85% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a, about 85% TF2a.

2. About 10% TF3, the remainder being Substantially only TF2a and TF2b: About 5% TF2a (the applicants point out for clarity, “about 5% TF2a” necessarily requires about 80% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a.

3. About 15% TF3, the remainder being Substantially only TF2a and TF2b:

About 5% TF2a (the applicants point out for clarity, “about 5% TF2a” necessarily requires about 75% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a.

4. About 20% TF3, the remainder being Substantially only TF2a and TF2b:

About 5% TF2a (the applicants point out for clarity, “about 5% TF2a” necessarily requires about 70% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a.

5. About 25% TF3, the remainder being Substantially only TF2a and TF2b:

About 5% TF2a (the applicants point out for clarity, “about 5% TF2a” necessarily requires about 65% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a.

6. About 30% TF3, the remainder being Substantially only TF2a and TF2b:

About 5% TF2a (the applicants point out for clarity, “about 5% TF2a” necessarily requires about 60% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a.

7. About 35% TF3, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

8. About 40% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

9. About 45% TF3, the remainder being Substantially only TF2a and TF2b:

About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

10. About 50% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

11. About 55% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

12. About 60% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

13. About 65% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

14. About 70% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

15. About 75% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

16. About 80% TF3, the remainder being Substantially only TF2a and TF2b:

About 5% TF2a (the applicants point out for clarity, “about 5% TF2a” necessarily requires about 10% TF2b), about 10% TF2a.

17. About 85% TF3, the remainder being Substantially only TF2a and TF2b:

About 5% TF2a (the applicants point out for clarity, “about 5% TF2a” necessarily requires about 5% TF2b).

B. About 10% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 80% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a, about 80% TF2a.

2. About 10% TF3, the remainder being Substantially only TF2a and TF2b:

About 5% TF2a (the applicants point out for clarity, “about 5% TF2a” necessarily requires about 75% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a.

3. About 15% TF3, the remainder being Substantially only TF2a and TF2b:

About 5% TF2a (the applicants point out for clarity, “about 5% TF2a” necessarily requires about 70% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a.

4. About 20% TF3, the remainder being Substantially only TF2a and TF2b:

About 5% TF2a (the applicants point out for clarity, “about 5% TF2a” necessarily requires about 65% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a.

5. About 25% TF3, the remainder being Substantially only TF2a and TF2b:

About 5% TF2a (the applicants point out for clarity, “about 5% TF2a” necessarily requires about 60% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a.

6. About 30% TF3, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

7. About 35% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

8. About 40% TF3, the remainder being Substantially only TF2a and TF2b: About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

9. About 45% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

10. About 50% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a

11. About 55% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

12. About 60% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

13. About 65% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

14. About 70% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

15. About 75% TF3, the remainder being Substantially only TF2a and TF2b:

About 5% TF2a (the applicants point out for clarity, “about 5% TF2a” necessarily requires about 10% TF2b), about 10% TF2a.

16. About 80% TF3, the remainder being Substantially only TF2a and TF2b:

About 5% TF2a (the applicants point out for clarity, “about 5% TF2a” necessarily requires about 5% TF2b).

C. About 15% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 75% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a, about 75% TF2a.

2. About 10% TF3, the remainder being Substantially only TF2a and TF2b:

About 5% TF2a (the applicants point out for clarity, “about 5% TF2a” necessarily requires about 70% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a.

3. About 15% TF3, the remainder being Substantially only TF2a and TF2b:

About 5% TF2a (the applicants point out for clarity, “about 5% TF2a” necessarily requires about 65% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a.

4. About 20% TF3, the remainder being Substantially only TF2a and TF2b:

About 5% TF2a (the applicants point out for clarity, “about 5% TF2a” necessarily requires about 60% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a.

5. About 25% TF3, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

6. About 30% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

7. About 35% TF3, the remainder being Substantially only TF2a and TF2b:

About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

8. About 40% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

9. About 45% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

10. About 50% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

11. About 55% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

12. About 60% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

13. About 65% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

14. About 70% TF3, the remainder being Substantially only TF2a and TF2b:

About 5% TF2a (the applicants point out for clarity, “about 5% TF2a” necessarily requires about 10% TF2b), about 10% TF2a.

15. About 75% TF3, the remainder being Substantially only TF2a and TF2b:

About 5% TF2a (the applicants point out for clarity, “about 5% TF2a” necessarily requires about 5% TF2b).

D. About 20% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 70% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a, about 70% TF2a.

2. About 10% TF3, the remainder being Substantially only TF2a and TF2b:

About 5% TF2a (the applicants point out for clarity, “about 5% TF2a” necessarily requires about 65% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a.

3. About 15% TF3, the remainder being Substantially only TF2a and TF2b:

About 5% TF2a (the applicants point out for clarity, “about 5% TF2a” necessarily requires about 60% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a.

4. About 20% TF3, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

5. About 25% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

6. About 30% TF3, the remainder being Substantially only TF2a and TF2b:

About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

7. About 35% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

8. About 40% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

9. About 45% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

10. About 50% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

11. About 55% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

12. About 60% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

13. About 65% TF3, the remainder being Substantially only TF2a and TF2b:

About 5% TF2a (the applicants point out for clarity, “about 5% TF2a” necessarily requires about 10% TF2b), about 10% TF2a.

14. About 70% TF3, the remainder being Substantially only TF2a and TF2b:

About 5% TF2a (the applicants point out for clarity, “about 5% TF2a” necessarily requires about 5% TF2b).

E. About 25% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 65% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a, about 65% TF2a.

2. About 10% TF3, the remainder being Substantially only TF2a and TF2b:

About 5% TF2a (the applicants point out for clarity, “about 5% TF2a” necessarily requires about 60% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a.

3. About 15% TF3, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

4. About 20% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

5. About 25% TF3, the remainder being Substantially only TF2a and TF2b:

About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

6. About 30% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

7. About 35% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

8. About 40% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

9. About 45% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

10. About 50% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

11. About 55% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

12. About 60% TF3, the remainder being Substantially only TF2a and TF2b:

About 5% TF2a (the applicants point out for clarity, “about 5% TF2a” necessarily requires about 10% TF2b), about 10% TF2a.

13. About 65% TF3, the remainder being Substantially only TF2a and TF2b:

About 5% TF2a (the applicants point out for clarity, “about 5% TF2a” necessarily requires about 5% TF2b).

F. About 30% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 60% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a, about 60% TF2a.

2. About 10% TF3, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

3. About 15% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

4. About 20% TF3, the remainder being Substantially only TF2a and TF2b: About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

5. About 25% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

6. About 30% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

7. About 35% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

8. About 40% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

9. About 45% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

10. About 50% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

11. About 55% TF3, the remainder being Substantially only TF2a and TF2b:

About 5% TF2a (the applicants point out for clarity, “about 5% TF2a” necessarily requires about 10% TF2b), about 10% TF2a.

12. About 60% TF3, the remainder being Substantially only TF2a and TF2b:

About 5% TF2a (the applicants point out for clarity, “about 5% TF2a” necessarily requires about 5% TF2b).

G. About 35% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 55% TF2b), about 10% TF2a, about 15% TF2a, about 20% TF2a, about 25% TF2a, about 30% TF2a, about 35% TF2a, about 40% TF2a, about 45% TF2a, about 50% TF2a, about 55% TF2a.

2. About 10% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

3. About 15% TF3, the remainder being Substantially only TF2a and TF2b:

About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

4. About 20% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

5. About 25% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

6. About 30% TF3, the remainder being Substantially only TF2a and TF2b: About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

7. About 35% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

8. About 40% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

9. About 45% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

10. About 50% TF3, the remainder being Substantially only TF2a and TF2b:

About 5% TF2a (the applicants point out for clarity, “about 5% TF2a” necessarily requires about 10% TF2b), about 10% TF2a.

11. About 55% TF3, the remainder being Substantially only TF2a and TF2b:

About 5% TF2a (the applicants point out for clarity, “about 5% TF2a” necessarily requires about 5% TF2b).

H. About 40% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 50% TF2b), about 10% TF2a, about 15% TF2a, about 20% TF2a, about 25% TF2a, about 30% TF2a, about 35% TF2a, about 40% TF2a, about 45% TF2a, about 50% TF2a.

2. About 10% TF3, the remainder being Substantially only TF2a and TF2b: About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

3. About 15% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

4. About 20% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

5. About 25% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

6. About 30% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

7. About 35% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

8. About 40% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

9. About 45% TF3, the remainder being Substantially only TF2a and TF2b:

About 5% TF2a (the applicants point out for clarity, “about 5% TF2a” necessarily requires about 10% TF2b), about 10% TF2a.

10. About 50% TF3, the remainder being Substantially only TF2a and TF2b:

About 5% TF2a (the applicants point out for clarity, “about 5% TF2a” necessarily requires about 5% TF2b).

I. About 45% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 45% TF2b), about 10% TF2a, about 15% TF2a, about 20% TF2a, about 25% TF2a, about 30% TF2a, about 35% TF2a, about 40% TF2a, about 45% TF2a.

2. About 10% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

3. About 15% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

4. About 20% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

5. About 25% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

6. About 30% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

7. About 35% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

8. About 40% TF3, the remainder being Substantially only TF2a and TF2b:

About 5% TF2a (the applicants point out for clarity, “about 5% TF2a” necessarily requires about 10% TF2b), about 10% TF2a.

9. About 45% TF3, the remainder being Substantially only TF2a and TF2b: About 5% TF2a (the applicants point out for clarity, “about 5% TF2a” necessarily requires about 5% TF2b).

J. About 50% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 40% TF2b), about 10% TF2a, about 15% TF2a, about 20% TF2a, about 25% TF2a, about 30% TF2a, about 35% TF2a, about 40% TF2a.

2. About 10% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

3. About 15% TF3, the remainder being Substantially only TF2a and TF2b: About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

4. About 20% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

5. About 25% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

6. About 30% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

7. About 35% TF3, the remainder being Substantially only TF2a and TF2b:

About 5% TF2a (the applicants point out for clarity, “about 5% TF2a” necessarily requires about 10% TF2b), about 10% TF2a.

8. About 40% TF3, the remainder being Substantially only TF2a and TF2b:

About 5% TF2a (the applicants point out for clarity, “about 5% TF2a” necessarily requires about 5% TF2b).

K. About 55% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 35% TF2b), about 10% TF2a, about 15% TF2a, about 20% TF2a, about 25% TF2a, about 30% TF2a, about 35% TF2a.

2. About 10% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

3. About 15% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

4. About 20% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

5. About 25% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

6. About 30% TF3, the remainder being Substantially only TF2a and TF2b:

About 5% TF2a (the applicants point out for clarity, “about 5% TF2a” necessarily requires about 10% TF2b), about 10% TF2a.

7. About 35% TF3, the remainder being Substantially only TF2a and TF2b:

About 5% TF2a (the applicants point out for clarity, “about 5% TF2a” necessarily requires about 5% TF2b).

L. About 60% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 30% TF2b), about 10% TF2a, about 15% TF2a, about 20% TF2a, about 25% TF2a, about 30% TF2a.

2. About 10% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

3. About 15% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

4. About 20% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

5. About 25% TF3, the remainder being Substantially only TF2a and TF2b:

About 5% TF2a (the applicants point out for clarity, “about 5% TF2a” necessarily requires about 10% TF2b), about 10% TF2a.

6. About 30% TF3, the remainder being Substantially only TF2a and TF2b:

About 5% TF2a (the applicants point out for clarity, “about 5% TF2a” necessarily requires about 5% TF2b).

M. About 65% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 25% TF2b), about 10% TF2a, about 15% TF2a, about 20% TF2a, about 25% TF2a.

2. About 10% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

3. About 15% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

4. About 20% TF3, the remainder being Substantially only TF2a and TF2b:

About 5% TF2a (the applicants point out for clarity, “about 5% TF2a” necessarily requires about 10% TF2b), about 10% TF2a.

5. About 25% TF3, the remainder being Substantially only TF2a and TF2b:

About 5% TF2a (the applicants point out for clarity, “about 5% TF2a” necessarily requires about 5% TF2b).

N. About 70% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 20% TF2b), about 10% TF2a, about 15% TF2a, about 20% TF2a.

2. About 10% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

3. About 15% TF3, the remainder being Substantially only TF2a and TF2b:

About 5% TF2a (the applicants point out for clarity, “about 5% TF2a” necessarily requires about 10% TF2b), about 10% TF2a.

4. About 20% TF3, the remainder being Substantially only TF2a and TF2b:

About 5% TF2a (the applicants point out for clarity, “about 5% TF2a” necessarily requires about 5% TF2b).

O. About 75% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 15% TF2b), about 10% TF2a, about 15% TF2a.

2. About 10% TF3, the remainder being Substantially only TF2a and TF2b:

About 5% TF2a (the applicants point out for clarity, “about 5% TF2a” necessarily requires about 10% TF2b), about 10% TF2a.

3. About 15% TF3, the remainder being Substantially only TF2a and TF2b:

About 5% TF2a (the applicants point out for clarity, “about 5% TF2a” necessarily requires about 5% TF2b).

P. About 80% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 10% TF2b), about 10% TF2a.

2. About 10% TF3, the remainder being Substantially only TF2a and TF2b:

About 5% TF2a (the applicants point out for clarity, “about 5% TF2a” necessarily requires about 5% TF2b).

Q. About 85% TF1, the remainder being Substantially only TF3, TF2a, and TF2b.

1. About 5% TF3, the remainder being Substantially only TF2a and TF2b:

About 5% TF2a (the applicants point out for clarity, “about 5% TF2a” necessarily requires about 5% TF2b).

### Polyphenols

This present invention further provides an improved process for economic manufacture of high quality theaflavins wherein tea polyphenols are extracted into an organic solvent, further extracted into organic solutions containing monomers of catechins and oxidized, for example, in the organic solvent by means of native enzymes directly from tea leaf material without using other source of polyphenols. The ranges of concentration for monomers of catechins in the organic solution prior to oxidation include, for example, 0.01-0.1% of EGC by weight, 0.25-2.5% of EGCG by weight, 0.02-0.2% of EC by weight, and 0.1-1% of ECG by weight.

In an alternative embodiment, the ranges of concentration for monomers of catechins in the organic solution prior to oxidation include 0.01-0.2% of EGC by weight, 0.1-5% of EGCG by weight, 0.01-0.4% of EC by weight, and 0.1-2% of ECG by weight. The fermentation process takes place in an organic phase, which not only improves the yield of theaflavins at low cost, but avoids problems such as emulsions in other manufacturing processes in multiphase media. The theaflavins extract is further purified by macroporous absorbent resin to obtain high purity of theaflavins. Process embodiments of the present invention employ, for example, neutral or weakly acidic conditions.

A process for producing modified theaflavins comprising grinding tea leaves to produce a stock fermentation enzyme, preparing a fermentation mixture by combining the stock fermentation enzyme and a modified substrate wherein the modified substrate comprises at least two catechins in a ratio different from that in tea leaves, and fermenting the fermentation mixture to produce crude theaflavins, purifying the crude theaflavins to produce modified theaflavins. An embodiment of this process is wherein the catechins in the modified substrate comprises at least 15% of Epicatechin (EC). An embodiment of this process is wherein the catechins in the modified substrate comprises at least 20% of Epigallocatechin (EGC). An embodiment of this process is wherein the catechins in the modified substrate comprises at least 20% of Epicatechin gallate (ECG). An embodiment of this process is wherein the catechins in the modified substrate comprises at least 50% of Epigallocatechin gallate (EGCG). An embodiment of this process is wherein the catechins in the modified substrate comprises EC and EGC in a ratio from 1:0.2 to 1:10. An embodiment of this process is wherein the catechins in the modified substrate comprises EC and EGCG in a ratio from 1:0.2 to 1:15. An embodiment of this process is wherein the catechins in the modified substrate comprises ECG and EGCG in a ratio from 1:0.2 to 1:15. An embodiment of this process is wherein the catechins in the modified substrate comprises ECG and EGC in a ratio from 1:0.2 to 1:15.

### Production

The term “macroporous absorbent resin” or “macroporous adsorption resin” includes, for example, H103 and D101 (Source: Shanghai Hualing Resin Co., Ltd.). As used herein the term organic solvent refers to any inert organic solvent that solubilizes tea leaf polyphenols including but not limited to esters, alcohols, ketones and ethers. The term inert as used herein refers to an organic solvent that does not otherwise affect the oxidation of polyphenols to theaflavins in the processes described herein. Esters for use as organic solvent in processes described herein include, but are not limited to, for example, methyl acetate, ethyl acetate, propyl acetate, and isopropyl acetate. Alcohols for use as organic solvent in processes described herein include, but are not limited to, for example, ethyl alcohol, propyl alcohol, isopropyl alcohol, iso-butanol, and n-butanol. Ketones for use as organic solvent in processes described herein include, but are not limited to, for example, acetone and butanone. Ethers for use as organic solvent in processes described herein include, but are not limited to, for example, diethyl ether, dipropyl ether, and methyl-tert-butyl ether.

This present invention provides an improved process for economic manufacture of high quality theaflavins wherein tea polyphenols are extracted into an organic solvent and oxidized, for example, in the organic solvent by means of native enzymes directly from tea leaf material without using other source of polyphenols. The fermentation process takes place in an organic phase, which not only improves the yield of theaflavins at low cost, but avoids problems such as emulsions in other manufacturing processes in multiphase media. Process embodiments of the present invention employ, for example, neutral or weakly acidic conditions.

An improved process for manufacturing theaflavins is described comprising the steps of extracting preferably large leaf tea with an organic solvent to produce an organic stock substrate solution, the solution may be treated to control the proportion of monomers of catechins to produce an organic substrate solution, preparing stock fermentation enzyme from tea leaves, and fermenting the organic stock substrate solution with the stock fermentation enzyme, for example, in an oxygenated environment to produce a fermentation mixture. Embodiments of the process described herein include filtering the resulting fermentation mixture and concentrating the resulting filtrate. Embodiments include washing the filtrate with an aqueous solution, then adding water to the washed filtrate and concentrating the resulting aqueous phase to produce aqueous theaflavin extract by removing, evaporating for example, remaining organic solvent. Embodiments include filtration of the aqueous theaflavin extract to remove insoluble impurities at this point to produce purified aqueous theaflavin extract. Embodiments include lyophilization or spray drying of the purified aqueous theaflavin extract to produce solid purified theaflavin extract. Example solid purified theaflavin extract compositions produce by the processes described herein contain about 45% theaflavins. Alternate example solid purified theaflavin extract compositions produce by the processes described herein contain about 80% theaflavins. Accordingly example solid purified theaflavin extract compositions produce by the processes described herein usually contain between about 40% and about 85% theaflavins. Example solid purified theaflavin extract compositions produced by the process described herein contain about 30% TF2a, about 10% TF2b, about 40% TF3, and less than about 3% TF1.

### Physiological Disorders

Treatment of mammalian physiological disorders are provided which comprise administering an effective amount of purified theaflavin extracts described herein. Method of control of a mammalian physiological disorder, particularly an influenza infection, is provided which comprises administering an effective amount of purified theaflavin extracts described herein to a mammal. Oral dosage forms are provided which comprise an effective amount of purified theaflavin extracts described herein. The term ‘effective amount’, as used herein, encompasses inter alia, an amount of theaflavin extract or individual dosage compositions described herein effective to control a mammalian physiological disorder. The term ‘controlling influenza infection’, as used herein, encompasses preventing or ameliorating infection or symptoms or physical conditions otherwise conferred by or associated with the influenza.

### Example Theaflavin Extract Characteristics Resulting from Processes Described Herein

TABLE 1

| Item | Specification | Note |
|---|---|---|
| Appearance | Pale yellow to red | |
| | brown color, astringent | |
| Total Theaflavins % | ≧80.0 | HPLC |
| Theaflavin (TF1) % | ≦4 | HPLC |
| Theaflavin-3-gallate (TF2a) % | 20-50% | HPLC |
| Theaflavin-3′-gallate (TF2b) % | 5-20% | HPLC |
| Theaflavin3,3′-digallate (TF3) % | 20-50% | HPLC |
| Water Content % | ≦6.0 | |
| Ash Content % | ≦2.0 | |
| Heavy Metal ppm | ≦10 | |
| Arsenic ppm | ≦2 | |
| Caffeine % | ≦4 | |

The present invention has a number of advantages over traditional processes. Because the fermentation process takes place in an organic phase and tea leaves, homogenized for example, are used as the source of fermentation enzyme, the manufacturing process is readily controllable and avoids problems such as emulsifications in traditional processes. Fermentation process also takes place in controllable manner, for example, adjusting the proportion of monomeric catechins in the organic stock substrate solution to produce theaflavins with specific proportion of mixture of monomeric theaflavins. In addition, this low cost manufacturing process produces theaflavins in high quality with a theaflavin content of up to 85% by weight determined by HPLC. The caffeine level, for example, is reduced to about 4% or less. The solubility, taste, and color of the product are significantly improved. Processes described herein, without using toxic solvent or generating large amount of waste, are environmentally friendly. Organic solvent is recycled, for example. Neutral or weakly acidic aqueous conditions are employed, for example, to enhance the quality of the product.

### An Exemplary Process of the Present Invention for the Production of Theaflavins Includes:

a. Preparation of fermentation stock solution (extracting of green leaves with an organic solvent): 500 Kg, for example, Green tea leaves are added into an extractor (Multifunctional Dynamic Extracting Tank; Model: ZY-DTQ-6.0; Manufacturer: Wenzhou Zhongyuan Light Industry Machine Co., for example), followed by the addition of 5000 Kg, for example, ethyl acetate. The mixture is stirred and extracted for 30 minutes at 10° C.-70° C., for example. The organic phase is collected. A second portion of 5000 Kg, for example, ethyl acetate is used for extracting the green tea leaves. The organic phases are combined, and concentrated at 0.06 Mpa, for example, at below 80° C., for example, (Scraped Evaporator, Model: ZYE-40; Manufacturer: Changshu Pharmaceutical & Chemical Equipment General Factory, for example) until a solid content of between about 0.5% and about 5% is reached; the concentration solution is washed in a water scrubber (Washing Vessel, Model: SX1000, Manufacturer: Changshu Pharmaceutical & Chemical Equipment General Factory, for example) and then water is added. The mixture is stirred up and then allowed to stand to separate into two layers. The bottom aqueous layer is removed. The remaining organic phase is washed 1-20 times using the same method as above. The purpose of this operation is to adjust proportion of monomeric catechins in the solution, then cooled to room temperature and used as the fermentation stock solution.
b. Preparation of fermentation enzyme: Fresh tea leaves 600 Kg, for example, are moisturized using pure water or 0.1%-1%, for example, of citric acid solution, frozen, and ground (40-100 Mesh, for example) as the fermentation enzyme.
c. Fermentation: 5000 L, for example, of fermentation stock solution obtained from step (a) is added to a fermentor (Model: FXG; Manufacturer: Changshu Pharmaceutical & Chemical Equipment General Factory, for example), followed by the addition of 500 Kg, for example, fermentation enzyme from step (b). The mixture in the fermentor undergoes fermentation process in the presence of gas, e.g., air with an agitation at 20° C.-40° C. The air flow rate is controlled at range of, for example, 0.2-20 m³/min, agitation speed, for example, 30-150 rpm. The ratio of fermentation stock solution and the enzyme by weight, for example, is between about 5:1 to about 20:1.
d. Separation: The fermentation solution from the step (c) is filtered through, for example, 120 mesh filter screen and divided into filtrate and filter residue.
e. Concentration: The filtrate from the step (d) is concentrated, for example, using Scraped Evaporator under conditions, for example, of relative vacuum 0.06 Mpa and temperature preferably below 80° C. (Model: ZYE-40; Manufacturer: Changshu Pharmaceutical & Chemical Equipment General Factory, for example) to solid content, for example, of 5%-30%.
f. Washing: The concentrated filtrate from the step (e) is put into water scrubber (Washing Vessel, Model: SX1000, Manufacturer: Changshu Pharmaceutical & Chemical Equipment General Factory, for example) and then the acidic solution is added. The mixture is stirred up and then allowed to stand to separate into two layers. The bottom aqueous layer is removed. The remaining organic phase is washed 1-20 times using the same method as above. The ratio of the acidic solution and concentrate by volume is about 0.1:1-1:1. After washing, the organic phase and aqueous phase are collected, respectively. The aqueous phase is concentrated and dried to form the by-product. The organic phase is used in the next step.
g. Solute transferring: Pure water is added into the organic phase solution obtained from the above step (f). The ratio of pure water and organic phase solution is bout 1:1-1:25 by weight. The organic phase and water are mixed thoroughly and concentrated at controlled temperature.
h. Column Separation: the concentrate from step (g) is flowed from the top of column with macroporous adsorbent resin, for example, H103 and D101 (source: Shanghai Hualing Resin Co., Ltd.), as a filling medium to further purify theaflavin extract. The separating process follows adsorption-elution cycle. The adsorption flow rate of the concentrate is 1-4 BV/h. The column is washed using washing solution (for example, 5%-10% ethyl alcohol aqueous solution) to remove the impurities, the flow rate of washing solution is controlled at, for example, 0.5-5 BV/h; the desorption solution is, for example, 20%-95% ethyl alcohol aqueous solution, the flow rate of desorption is controlled at, for example, 0.5-5 BV/h; the temperature of the entire adsorption-elution cycle is controlled at, for example, 0° C.-60° C. The ratio of diameter and height of the column is about, for example, 1:4 to about 1:25; eluate from desorption is monitored based on emerge of TF2a (analyzed by using HPLC). The eluate of the first phase (no theaflavins) is collected for producing other by-products, and the eluate of the second phase, that contains high contents of TF2a, TF2b and TF3 is collected for further treatment.
i. Alternative Column Separation: The concentrate from step (g) is optionally re-dissolved with one or more solvents suitable, for example ethanol, before being loaded to a chromatographic column. The chromatographic column is packed with suitable material, for example, silica gel. A UV detector, for example, a Waters 486 UV/VIS detector, is used for monitoring the purification process. The column is eluted with a suitable eluent, for example, 40% ethyl acetate/hexane to provide fractions containing TF1, TF2a, TF2b, TF4, individually or in combination.
j. Filtration: The concentrate obtained from step (g) is cooled to 30° C.-60° C., and filtered. The filtrate is collected.
k. Concentrating and freeze-drying: The filtrate obtained from the step (h) is further concentrated to solid content of 15%-80%, for example, by using High Efficiency Rotary Scraped Evaporator (Model: DN1200; Manufacturer: Wuxi Xuelang Fermentation Equipment Co., Ltd.) at relative vacuum 0.085 Mpa, for example, and temperature below 80° C., for example. The concentrate is subjected to spray drying, or lyophilization, then ground using high speed mill, for example, and screened using screen grader, for example, to obtain the product of theaflavins.

Exemplary modifications of processes of the present invention, exemplified supra, include embodiments wherein the ratio of tea leaves, preferably large leaf tea, and ethyl acetate each time in step (a) above for the preparation of fermentation stock solution is within the range of about 1:10 to about 1:30. The example process of preparation of enzyme in step (b) is performed at below 0° C., for example, between about −10° C. and 0° C., for at least about 2 hours. The size of ground fresh tea leaves is preferably about 80 mesh or smaller. Gas introduced into the fermentation process in step (c) is preferably pure oxygen. The temperatures in the concentration (step (e)) and transferring solute (step (g)) are controlled at below 80° C., for example between about 60° C. and about 80° C.]. The acidic solution added in washing process in step (f) is about 0.05% to about 1% of aqueous citric acid. The temperatures of the lyophilization process for concentration in step (i) are preferably controlled at below about −35° C. to form an ice block. The vacuum level is preferably about 0.6-1 mmHg while water vapor is evaporated from the ice block. The temperature is then increased, for example, at a rate of 3° C./hour to 0° C., then, for example, at a rate of 5° C. to 25° C.-30° C. and stayed, for example, for 1-2 hours until the water content of the product reaches about 3% to about 5%. The vacuum is removed to obtain, for example, a theaflavins product of the present invention.

Preferred embodiment processes of the present invention are wherein the controlled temperature is between 25-60° C. for extracting green tea leaves. A further example is wherein the fermentation stock solution and said fermentation enzyme are in a ratio of about 5:1 to about 20:1 by weight. Another example process feature is wherein said second controlled temperature is between 20-40° C. Solid product may be obtained by means of crystallization or lyophilization, for example.

### Preparation of Fermentation Stock Solution (Organic Stock Substrate Solution) for Fermentation Enzymatic Oxidation:

To a container, green tea leaves, preferably shredded into 5-60 mesh, are added, followed by the addition of ethyl acetate. The ratio of green tea leaves and ethyl acetate added is between about 1:10 and about 1:30 by weight. The mixture is stirred and extracted for 30 minutes, for example, at 25° C.-60° C. The extracted solution is collected by filtration or decantation. To the resulting solid residue ethyl acetate is added. The ratio of solid residue and ethyl acetate at this point is between about 1:10 and about 1:30 by weight. The extraction procedure is repeated to yield a second extraction. Extracted ethyl acetate solutions are combined to one container, concentrated at vacuum 0.085-0.054 Mpa at 56-80° C. in a rotating evaporator (e.g., odel: RE-200, Shanghai Kexing Instrument Co.) to solid content in the range of 0.5%-5%, and cooled to room temperature as the fermentation stock solution (organic stock substrate solution).

Preparation of fermentation Enzyme (stock fermentation enzyme), e.g., polyphenol oxidase and polyphenol peroxidase: Fresh tea leaves are moistened with pure water or 0.1%-1% of citric acid aqueous solution, frozen for at least about 2 hours, for example, then ground and filtered through a screen, preferably of at least about 80 mesh, resulting in an example stock fermentation enzyme.

Fermentation process: The fermentation stock solution (organic stock substrate solution) is added to a fermentor (fermentation container), followed by the addition of the fermentation enzyme (stock fermentation enzyme). A preferred ratio of fermentation stock solution and the enzyme is between about 5:1 and about 20:1 by weight. Gas, preferably pure oxygen, is introduced into the resulting mixture in the fermentor while stirred at about 30- to about 150 rpm. The fermentation (enzymatic oxidation) is preferred to proceed at about 20° C. to about 40° C.

Separation: The fermentation solution mixture is separated (theaflavins from the fermentation mixture to produce purified theaflavin extract) by filtration, for example, and divided into filtrate and solid residue.

Concentration: The filtrate (theaflavin extract) is concentrated on a rotating evaporator (e.g. rotating evaporator (Model: RE-200, Manufacturer: Shanghai Kexing Instrument Co.) at controlled temperature to yield a mixture with about 5% to about 30% solid content.

Washing: Concentrated mixture is transferred to a water scrubber (e.g., Pear Shaped Separatory Funnel, 500 ml, Manufacturer: Shanghai Shendi Glassware Co.) followed by acidic solution, preferably 0.05%-1% citric acid aqueous solution. The ratio of the acidic solution and concentrated mixture by volume is between about 0.1:1 and about 1:1. The two phases (aqueous and organic) are mixed thoroughly and were allowed to stand to separate.

The bottom layer, i.e., water layer, is removed. The remained top layer is washed 1-20 times using the same method, i.e. addition of acidic solution while being stirred and mixed, separation of two layers and removal of the bottom water layer. The ratio of the acidic solution and concentrate by volume is about 0.1:1 to about 1:1. After washing, the organic phase and aqueous phase are collected, respectively. The aqueous phase is concentrated and dried to form the product. The organic phase is further processed.

Pure water is added to the organic solution obtained. The ratio of pure water and ester organic phase solution is between about 1:1 and about 1:25 by weight. This is concentrated at controlled temperature, preferably at or below 80° C.

Filtration: the concentrate solution obtained from step G is cooled to 30° C.-60° C., then filtered. The filtrate is collected.

Concentration and Drying: Filtrate obtained from step (h) is further concentrated to the solid content of 15%-80%. The resulting concentrate is dried by lyophilization, or spray drying, ground, and screened to obtain product theaflavins.

Preferred features of the process described herein include temperatures of the concentration/lyophilization is controlled at −35° C. or below, the vacuum level is about 0.6-1 mmHg. The water vapor is evaporated from the ice block particles of theaflavins concentrate, then, the temperature is increased at the rate of 3° C./hour to 0° C., then the temperature is increased at the rate of 5° C. to 25° C.-30° C., keeping for 1-2 hours until the water content of the product reaches to 3%-5%, and finally, the vacuum is released.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” means the amount of a compound that, when administered to a patient for treating or preventing a disease, is sufficient to effect such treatment or prevention of the disease. The “therapeutically effective amount” will vary depending on the disease and its severity and the age, weight, etc., of the patient to be treated.

Further, in certain embodiments, compounds of the invention and/or pharmaceutical compositions thereof are administered to a patient, preferably a human, as a preventative measure against the above diseases or conditions. Thus, the theaflavins of the invention and/or compositions thereof may be administered as a preventative measure to a patient having a predisposition for any of the above diseases or disorders. Accordingly, the theaflavins of the invention and/or pharmaceutical compositions thereof may be used for the treating or preventing one disease or disorder and concurrently treating or preventing another (e.g., preventing hyperlipidemia while treating a cerebral infarction).

The suitability of the theaflavins of the invention and/or compositions thereof in treating or preventing the various diseases or disorders listed above may be determined by methods described in the art. Accordingly, it is well within the capability of those of skill in the art to assay and use the compounds of the invention and/or pharmaceutical compositions thereof to treat or prevent the above diseases or disorders.

### Therapeutic/Prophylactic Administration

The theaflavins of the invention and/or compositions thereof may be advantageously used in human and veterinary medicine.

When used to treat or prevent the above diseases or disorders, theaflavins of the invention and/or compositions thereof may be administered or applied singly, or in combination with other agents. The compounds of the invention and/or pharmaceutical compositions thereof may also be administered or applied singly, in combination with other pharmaceutically active agents including other compounds of the invention and/or pharmaceutical compositions thereof.

The current invention provides methods of treatment and prophylaxis by administration to a patient of a therapeutically effective amount of a compound of the invention and/or pharmaceutical composition thereof. The patient is preferably a mammal and most preferably, is a human.

The compounds of the invention and/or pharmaceutical compositions thereof are preferably administered orally, which results in the release of the compounds of the invention and/or pharmaceutical compositions thereof into the bloodstream. The compounds of the invention and/or pharmaceutical compositions thereof can be delivered via oral sustained release systems. In one embodiment, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropyl methylcellulose). Other preferred cellulose ethers have been described (Alderman, Int. J. Pharm. Tech. & Prod. Mfr. 1984, 5 (3) 1-9). Factors affecting drug release are well known to the skilled artisan and have been described in the art (Bamba et al., Int. J. Pharm. 1979, 2, 307).

In another embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In still another embodiment, osmotic delivery systems are used for oral sustained release administration (Verma et al., Drug Dev. Ind. Pharm., 2000, 26:695-708). In still another embodiment, OROS™ osmotic devices are used for oral sustained release delivery devices (Theeuwes et al., U.S. Pat. No. 3,845,770; Theeuwes et al., U.S. Pat. No. 3,916,899).

In one embodiment, the compounds of the invention are encapsulated for oral administration. Preferably, encapsulation protects the compounds of the invention from light and/or oxygen degradation. The capsule preferably, is comprised of a shell and a fill material, where either the shell or fill material contains an radiation blocker and/or an anti-oxidant.

The shell material is comprised of a gelling agent, water and optionally a plasticizer. Accordingly, the shell material may form either a hard gel or a soft gel. The gelling agent may be, but is not limited to, gelatin, modified starch, carrageenan, gellan, mannan gum, amylose, xanthan, alginates, agar, guar, gum arabic, pectin, cyclodextrin or combination thereof. The shell may optionally include an emulsifier, thickener, preservative, flavoring, sweetener, colorant, radiation blocker, opacifying agent, anti-oxidant, masticatory substance, etc.

Gelatin, as is well known in the art, is manufactured by the hydrolysis of animal by-products such as bones, skin, and connective tissue which contain collagen. Bovine animals and pigs are the primary sources of gelatin

Modified starches, include, for example, non-retrograding starches derived by chemical modification of starch from any plant source such as corn, waxy maize, potato, wheat, rice, tapioca, sorghum, etc. Useful modified starches are ether and ester derivatives of starch including, for example, hydroxypropyl, hydroxyethyl, succinate, and octenyl succinate starch derivatives. Other modified starches which may be used include the thermally converted, fluidity or thin boiling type products derived from the above chemically modified starches. These materials may be of lower molecular weight, prepared by heating the modified starch, subjecting the starch to hydrolytic acid and/or heat treatment, etc.

Carrageenan is a natural sulfated polysaccharide hydrocolloid derived from seaweed, and is a mixture of galactose and 3-6-anhydrogalactose copolymers. A number of different carrageenan types exist (e.g., kappa, iota, lambda, etc.) and it is anticipated that any of these may be used in the present invention.

Gellan gum is an extracellular polysaccharide obtained by aerobic fermentation of the microorganism, Pseudomonas elodea. Various forms of gellum gum including, but not limited to, native, deacetylated, deacylated clarified, partially deacetylated, partially deacylated clarified may be sued in the present invention.

Mannam gum include the galactomannan gums, the glucomannan gums and mixtures thereof.

Accordingly, mannam gum includes, but is not limited to, locust bean gum, konjac gum, tara gum and cassia gum.

In some embodiments, a gelling salt may be used in the present invention. Accordingly, a calcium salt, a magnesium salt, a barium salt, a sodium salt or a potassium salt of an appropriate inorganic or organic acid may be used to form the shell of a capsule of the present invention.

Plasticizers are preferably, polyols, such as, for example, glycerin, sorbitol, an alkylene glycol, maltitol, lactitol, xylitol, corn syrup solids, etc. and combinations thereof. In one embodiment, the plasticizer is a combination of glycerin and sorbitol.

In another embodiment, the capsule shell comprises between about 10% and 90% gelatin, and between about 5% and about 40% water. In another embodiment, the capsule shell comprises between about 10% and 90% gelatin, between about 1% and about 30% plasticizer and between about 5% and about 40% water. In still another embodiment, the capsule shell comprises between about 25 to about 45% gelatin, between about 1% and about 30% plasticizer and between about 5% and about 40% water. In still another embodiment, the capsule shell comprises between about 25 to about 45% gelatin, between about 1% and about 30% plasticizer, between about 1 and 5% radiation blocker and between about 1% and about 5% colorant and between about 5% and about 40% water.

The capsule shell of the present invention encloses a pre-selected quantity of fill material. Preferably, the enclosed fill material will contain a therapeutically effective amount of a mixture of theaflavins. The fill material may be a liquid, a semi-solid, a solid and gel.

The fill material may include a pharmaceutically acceptable vehicle, which may be prepared by a number of diverse methods which are known to those of skill in the art. The pharmaceutically acceptable vehicle must be compatible with the capsule shell and preferably, does not degrade the capsule shell during a period of typical storage. Solutions to the above problem are well known to the skilled artisan.

Suitable liquid pharmaceutically acceptable vehicles for the fill material include, but are not limited to, neutral oil, mineral oil, water, alcohol, polyalkylene glycol, vegetable oil and fructose syrup. Preferably, the liquid pharmaceutically acceptable vehicle is a vegetable oil, more preferably, a corn oil, peanut oil, safflower oil, sunflower oil and soybean oil and most preferably soybean oil. The liquid pharmaceutically acceptable vehicle may include an emulsifier, thickener, preservative, flavoring, sweetener, colorant, radiation blocker, opacifying agent, anti-oxidant, masticatory substance, etc.

The present capsules may also include a solid fill material. Useful solid fill materials include, but are not limited to, tablets or pellets comprising the mixture of theaflavins, which may be further coated with gelatin, sugar, etc. (Glassman, U.S. Pat. No. 3,228,789). The tablets or pellets may contain co-solvents, buffers, emulsifiers, thickeners, preservatives, sweeteners, flavorings, colorants, radiation blockers, anti-oxidants, opacifying agent, masticatory substances, etc.

The present capsules may also include a semi-solid fill material. The mixture of theaflavins may, for example, be dispersed in a substantially water-free carrier mixture comprising one or more polyalkylene glycols, preferably, comprising a mixture of liquid polyalkylene glycol and waxy polyalkylene glycol and in minor amount a C2-C.sub.4 diol or triol (Shah et al., U.S. Pat. No. 4,935,243). A flavoring, preservative, sweetener, colorant, radiation blocker, co-solvent, buffer, emulsifier, thickener, anti-oxidant, opacifying agent, masticatory substance, etc., may be added to the semi-solid fill material.

The present capsules may also enclose a gel fill comprising a gelled polymeric matrix, which may be generated by gelling a liquid fill followed by encapsulation (Cohen et al, U.S. Pat. No. 4,708,834). The gelled fill may comprise a solution or dispersion of an active ingredient in a polysaccharide gum and may also optionally, include co-solvents, preservative, buffers, emulsifiers, thickeners, sweeteners, flavorings, colorant, radiation blocker, opacifying agent, anti-oxidant, masticatory substance, etc.

Masticatory substances, for example, include, but are not limited to, Chicle, Chiquibul, Crown gum, Gutta hang kang, Massaranduba balata, Massaranduba chocolate, Nispero, Leche caspi, Pendare, Perillo, Leche de vac, Niger gutta, Tunu, Chite and Natural rubber. Flavorings include, but are not limited to, cherry syrup, citric acid, dextrose, essential oil, vanillin, cinnamon oil, orange oil, spearmint oil, strawberry oil, nutmeg oil. A preferred stiffener is beeswax while a preferred emulsifier is lecithin. Other stiffeners and emulsifiers which may be useful in the present invention are known to the skilled artisan. Preferred anti-oxidants include ascorbic acid and vitamin E.

In one embodiment, the fill material comprises between about 1% and about 20% mixture of theaflavins, between about 1% and about 5% anti-oxidant, between about 5% and about 90% pharmaceutically acceptable carrier, between about 1% and about 20% emulsifier; and between about 1% to about 20% stiffening agent.

### Oral Dosage Compositions

Exemplary oral dosage compositions typically contain a therapeutically effective amount of a compound of the invention, preferably in purified form, together with a suitable amount of a pharmaceutically acceptable vehicle, so as to provide the form for proper administration to a patient. The compounds of the invention may be present at a level of between about 5% and about 50% (w/w), preferably, about 11% in a pharmaceutical composition, for example. Total amount of the compound of the invention per dose may be between about 70 mg and about 210 mg.

When administered to a patient, the compounds of the invention and pharmaceutically acceptable vehicle are preferably sterile. Water, saline solutions and aqueous dextrose and glycerol solutions may be employed as liquid vehicles. Other suitable pharmaceutical vehicles include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents or pH buffering agents. In addition, auxiliary, stabilizing, thickening, lubricating and coloring agents may be used. A general discussion of the preparation of pharmaceutical compositions may be found in Remington, “The Science and Practice of Pharmacy,” 19th Edition.

Pharmaceutical compositions comprising a compound of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds of the invention into preparations which can be used pharmaceutically.

The present pharmaceutical compositions can take the form of solutions, suspensions, emulsion, tablets, pills, pellets, capsules, capsules containing liquids, powders, sustained-release formulations, emulsions or any other form suitable for oral use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington, “The Science and Practice of Pharmacy,” 19th Edition, 1995). Orally administered pharmaceutical compositions may contain one or more optional agents, for example, sweetening agents such as fructose, aspartame or saccharin; flavoring agents such as peppermint, oil of wintergreen, or cherry coloring agents and preserving agents, to provide a pharmaceutically palatable preparation. Moreover, when in tablet or pill form, the pharmaceutical compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5.0 mM to about 50.0 mM), etc. Additionally, flavoring agents, preservatives, coloring agents, bile salts, acylcamitines and the like may be added. For buccal administration, the pharmaceutical compositions may take the form of tablets, lozenges, etc. formulated in conventional manner.

When a compound of the invention is acidic or basic, it may be included in any of the above-described formulations as the free acid or free base, a pharmaceutically acceptable salt, a solvate or hydrate. Pharmaceutically acceptable salts substantially retain the activity of the free acid or base, may be prepared by reaction with bases or acids and tend to be more soluble in aqueous and other protic solvents than the corresponding free acid or base form.

Theaflavins of the invention and/or pharmaceutical composition thereof, will generally be used in an amount effective to achieve the intended purpose. For use to treat or prevent diseases or disorders the compounds of the invention and/or pharmaceutical compositions thereof, are administered or applied in a therapeutically effective amount.

The amount of a compound of the invention and/or pharmaceutical composition thereof that will be effective in the treatment of a particular disorder or condition disclosed herein will depend on the nature of the disorder or condition, and can be determined by standard clinical techniques known in the art and by doctors skilled in treating or preventing a particular disease or disorder. In addition, in vitro or in vivo assays may optionally be employed to help identify optimal dosage ranges. The amount of a compound of the invention and/or pharmaceutical composition thereof administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration, by multiple applications or controlled release. In one embodiment, the compounds of the invention are delivered by oral sustained release administration. Preferably, in this embodiment, the compounds of the invention are administered twice per day (more preferably, once per day). Dosing may be repeated intermittently, may be provided alone or in combination with other drugs and may continue as long as required for effective treatment of the disease state or disorder.

Suitable dosage ranges for oral administration are dependent on the nature of the compounds of the invention administered (e.g., whether the theaflavins are administered together or whether theaflavin, theaflavin-3-gallate, theaflavin-3′-gallate or theaflavin-3,3′-digallate are administered, each as a separate compound) but are generally about 0.001 mg to about 200 mg of a compound of the invention per kilogram body weight. In one embodiment, the dosage range is between about 0.1 mg/kg to about 5 mg/kg. Dosage ranges may be readily determined by methods known to the artisan of ordinary skill. Effective doses may be extrapolated from dose-response curves derived from in vitro or animal model test systems. Such animal models and systems are well-known in the art.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred. The compounds of the invention may also be demonstrated to be effective and safe using animal model systems.

Preferably, a therapeutically effective dose of a compound of the invention and/or pharmaceutical composition thereof described herein will provide therapeutic benefit without causing substantial toxicity. Toxicity of compounds of the invention and/or pharmaceutical compositions thereof may be determined using standard pharmaceutical procedures and may be readily ascertained by the skilled artisan. The dose ratio between toxic and therapeutic effect is the therapeutic index. A compound of the invention and/or pharmaceutical composition thereof will preferably exhibit particularly high therapeutic indices in treating disease and disorders. The dosage of a compound of the invention and/or pharmaceutical composition thereof described herein will preferably be within a range of circulating concentrations that include an effective dose with little or no toxicity.

In certain embodiments of the present invention, the compounds of the invention and/or pharmaceutical compositions thereof can be used in combination therapy with at least one other therapeutic agent. The compound of the invention and/or pharmaceutical composition thereof and the therapeutic agent can act additively or, more preferably, synergistically. In a preferred embodiment, a compound of the invention and/or a pharmaceutical composition thereof is administered concurrently with the administration of another therapeutic agent. In another embodiment, a compound of the invention and/or pharmaceutical composition thereof is administered prior or subsequent to administration of another therapeutic agent.

In particular, in one preferred embodiment, the compounds of the invention and/or pharmaceutical compositions thereof can be used in combination therapy with other agents used to treat or prevent hyperlipidemia, coronary heart disease, apoplexy, arthesclerotic cardiovascular diseases, AIDS, diabetes, oxidated-low density lipoprotein level, von Willebrand's disease, leukopenia, cerebral infarction, dementia and physical disorder and fatty liver.

### Diet Supplement Compositions

Exemplary diet supplement compositions typically contain one or more compounds of the invention, preferably in purified form, together with a suitable amount of a diet supplement vehicle, so as to provide the form for proper administration to a user.

There are many types of nutrition bars and other “snack” bars available on the market, and many consumers use such products as a convenient food source. For example, grain based bars such as granola bars are easy to carry and provide a healthy, good tasting food that is consumed by active people such as hikers and athletes, and by everybody else. Because grain-based nutrition bars are convenient and healthy, they have become a very popular product.

One type of bar that has become popular in the recent years is commonly called an “energy bar” or “performance bar.” U.S. Pat. No. 7,247,336, for example is herein incorporated by reference. U.S. Pat. No. 6,143,335, McKenzie, R. G., for example, teaches scoring a food bar into bite-sized pieces thus providing a method for delivering exact quantities of supplemental ingredients to animals or humans. These products are typically especially formulated for use by active individuals such as athletes, and include ingredients that are intended to boost athletic performance, endurance, etc. Such energy bars provide an easy way for athletes to consume foods that are especially formulated to improve performance.

### EXAMPLES

### Example I

### Study of Neuraminidase (NA) Inhibitory Activity of Theaflavins

### Materials and Reagents:

1. Theaflavins (60%), green tea polyphenols (98%) and epigallocatechin gallate (EGCG) extracted from tea leaf (Camellia sinensis), are manufactured and provided by Jiangsu Dehe Bio-Tech Co., Ltd., China;
2. 14077, 14078 and 14079 are compounds from Center for National Drug Screening, Beijing, China;
3. Oseltamivir is provided by Center for National Drug Screening, Beijing, China;
4. 4-MUNANA is a substrate of neuraminidase for determining its activity;
5. Influenza virus strain A/PR/8/34 (H1N1) is from Center for Diseases and Control, Beijing, China;
6. Cell: Madin-Darby canine kidney (MDCK) is from Center for National Drug Screening, Beijing, China.

Compounds tested and neuraminidase (NA) are suspended in the buffer solution (pH 6.5), then the fluorogenic substrate is added into the above reaction system to initiate the reaction, incubated for 60 minutes at temperature of 37° C. Fluorescence intensity is measured at the condition of excitation wavelength of 365 nm and emission wavelength of 455 nm. The fluorescence intensity of the reaction system inflects activity of NA enzyme. The inhibition rate on NA activity is calculated based on the reduced amount of fluorescence intensity. The inhibition rate of NA is expressed as follows:

Inhibition Rate of NA=(F_{Control Enzyme}−Fₛₐₘₚₗₑ)/(F_{Control Enzyme}−F_{substrate})×100%

### Results:

Table 2 shows inhibition effect of theaflavins on activity of neuraminidase (NA) of influenza virus (H1N1).

TABLE 2
Inhibitory Activity of Theaflavins on NA of Influenza Virus (H1N1)

| | Concentration | Inhibition | IC50 |
|---|---|---|---|
| Compounds | (μg/ml) | Rate (%) | (μg/ml) |
| 14077 | 40 | 76.92 | 26.09 |
| 14078 | 40 | 70.55 | 29.10 |
| 14079 | 40 | 79.00 | 26.18 |
| Theaflavins | 40 | 65.27 | 30.28 |
| Tea polyphenols | 40 | 57.64 | 33.83 |
| EGCG | 40 | 54.96 | 35.95 |
| Oseltamivir | 0.4 | 93.08 | 0.034 |

### Conclusions:

The experiment results indicate that theaflavins show substantial inhibition activity on influenza virus (H1N1), fifty percent inhibitory concentrations (IC₅₀) of theaflavins from Jiangsu Dehe Bio-Tech Co., Ltd. is 30.28 g/ml, inhibition rate of theaflavins on NA of H1N1 is 65.27%. Although the data from Table 2 shows that theaflavins is not as potent NA inhibitor of H1N1 as positive control of Oseltamivir (IC₅₀ and Inhibition Rate are 0.034 and 93.08%, respectively), theaflavins, as a natural and safe component from tea, exhibit substantial effects of antiviral activity by inhibiting NA activity and thus confering the ability to control and prevent influenza diseases.

### Example II

### Inhibitory Effect of Theaflavins on Cytopathic Effects (CPE) Induced by Influenza Virus (H1N1)

### Materials and Reagents:

1. Influenza Virus Strain: A/PR/8/34 (H1N1), replicated by from Center for Diseases and Control, Beijing, China
2. Cell Strain: Madin-Darby canine kidney (MDCK), from Center for National Drug Screening, Beijing, China.
3. Culture Media: DMEM Culture Media (purchased from Gibco) with 10% serus
4. Maintaining Culture Media: DEME culture media with 5 μg/ml of trysogen

### Method:

1×10⁵/ml MDCK cells per well were seeded in 96-well plate in 1001 of culture media and incubated 24 hours at 37° C., 5% CO₂. Cell monolayers were formed. Maintaining culture media was washed one time with PBS, and then culture containing A/PR/8/34 (H1N1) virus, maintaining culture, maintaining culture containing testing compounds, and culture containing testing compounds and virus were added, respectively, incubated for 24 hours at 37° C., 5% CO2. Cell activity was measured by crystal violet staining method. Inhibitory rate of Cell Pathological Changes (CPE) is calculated as:

Inhibitory Activity=(ASample−AModel)/(AControl−AModel)×100%

### Results:

TABLE 3
Inhibitory Activity of Theaflavins & Its Derivatives on
H1N1 Virus in Cellular Level

| | Concen- | Inhibitory | | | |
|---|---|---|---|---|---|
| Com | tration | Rate | IC50 | CC50 | CC50/ |
| pounds | (μg/ml) | (%) | (μg/ml) | (μg/ml) | IC50 |
| Teaflavins | 12.5 | 79.09 ± 25.34 | 8.72 ± 4.05 | >100 | >11.47 |
| Tea | 50 | 87.22 ± 1.03 | 30.91 ± 4.05 | >100 | >3.24 |
| poly- | | | | | |
| phenols | | | | | |
| EGCG | 50 | 56.85 | 42.05 | >100 | >2.38 |
| 14077 | 25 | 97.26 ± 3.88 | 12.38 ± 7.75 | >100 | >8.08 |
| 14078 | 25 | 100 | 7.26 ± 2.19 | >100 | >13.77 |
| 14079 | 25 | 100 | 7.23 ± 3.16 | >100 | >13.83 |

### Conclusions:

The data in Table 3 shows that theaflavins and their derivatives exhibit inhibitory activity of Cell Pathological Changes (CPE), theaflavins' inhibitory rate of CPE is 79.09±25.34%; 50% inhibitory concentration (IC50) of theaflavins is lower than green tea polyphenols and EGCG, suggests that theaflavins has stronger inhibitory effect on virus in cellular level. The concentration of compound with 50% cytotoxic effect (CC50) for all of testing compounds is >100, exceeded the measuring limit, that indicates all of testing compounds are exhibited very low cytotoxicity. The Therapeutic Index (TI) is expressed as CC50/IC50, is a common indicative of the selectivity of a compound and consequently its effectiveness and usability. Therefore, based on theaflavins's low cytotoxicity (CC50) and high TI (CC50/IC50), theaflavins and their derivatives show certain antivirus effects at both enzymatic and cellular levels.

### Example III

### Evaluation of Neuraminidase (NA) Inhibitory Activity

### Materials and Methods:

1. Samples: Theaflavins 60% (TF60%) and theaflavins 80% (TF80%); provided by Jiangsu Dehe Biotechnology Co., Ltd.; Sample Receiving Date: Feb. 1, 2010
2. Other materials and reagents are same as the Study I.

### Methods:

In enzymatic reaction system, a certain concentration of testing compound and neuraminidase from influenza virus are suspended in buffer solution of the reaction (pH 6.5), then the fluorogenic substrate of MUNANA is added to initiate the reaction, it is incubated for 60 min at 37° C., and the stopper solution is added to terminate the reaction. Under the conditions of excitation wavelength 365 nm and emission wavelength 455 nm, the fluorescence intensity is measured. Fluorescence intensity of the reaction system reflects the enzymatic activity. According to the decreased amount of fluorescence intensity, the inhibitory rate of testing compound against NA activity can be calculated.

### Results:

The results of NA inhibitory activity of theaflavins are shown in Table 4.

TABLE 4
NA (H1N1) Inhibitory Activity of Theaflavins

| | Concentration | Inhibitory | |
|---|---|---|---|
| Sample | (μg/ml) | Rate % | IC50 (μg/ml) |
| TF60% | 40 | 64.24 ± 1.32 | 28.88 ± 2.32 |
| TF80% | 40 | 81.20 ± 2.53 | 23.63 ± 2.09 |
| Zanzmivir | 0.004 | 93.75 | 2.40 × 10⁻⁴ |

### Conclusions:

In the screening model of neuraminidase inhibitor, theaflavins 60% and theaflavins 80% all percent certain neuraminidase inhibitory activity, their IC₅₀ are 28.88±2.32 μg/ml and 23.63±2.09 μg/ml, respectively. Their NA inhibitory activities are lower than positive control drug.

### Example IV

### Evaluation of Inhibitory Activity of Cytopathic Effects (CPE) Induced by Influenza Virus

### Materials and Reagents:

1. Samples: Theaflavins 60% (TF60%) and theaflavins 80% (TF80%); provided by Jiangsu Dehe Biotechnology Co., Ltd.; Sample Receiving Date: Feb. 1, 2010
2. Other materials and reagents are same as the Study 1.

### Methods:

Cell: Madin-Darby canine kidney (MDCK). The culture is DMEM culture with 10% serum (Purchased from Gibco). The maintaining culture is non-scrum DMEM culture with 5 μg/ml trypsogen, and is used for maintaining culture after virus infection.

MDCK cell in logarithmic phase is added to DMEM culture that contains 10% serum, 1×10/ml of MDCK cell mixture is added into the culturing plate with 96 wells, and incubated in 5% CO₂ incubator for 24 hours at temperature of 37° C., the single cell layer is formed. The samples of TF80% and TF60% are diluted by DMEM culture, respectively, to different concentrations for later use. In the experiment, the following groups are designated: experimental group, regular control group and virus infected control group. The subtype of H1N1 influenza virus A/PR/8/34 (200TCIDDD₅₀) is added to MDCK cell, the virus is absorbed for 1 hour at 33° C., then it is taken out, and virus is removed and then added to experimental cultures of different concentrations. They are incubated for 24 hours at temperature of 33° C. and 5% CO₂. Taking out and adding 1% staining solution of crystal violet ammonium oxalate to stain cells for 10 min, washing 96 holes culture plate using water, then drying. 100 μl of 1% SDS is added to each well, vibrate, and measuring the cellular activity at 570 nm wavelength.

### Results:

The results of virus (H1N1) inhibitory activity of theaflavins at cellular level are shown in Table 5.

TABLE 5
Virus (H1N1) Inhibitory Activity of Theaflavins at Cellular Level

| Sample | MNCC (μg/ml) | CC50 (μg/ml) | IC50 (μg/ml) | CC50/IC50 |
|---|---|---|---|---|
| TF60% | 25 | >100 | 34.43 ± 11.57 | >2.9 |
| TF80% | 25 | >100 | 27.39 ± 9.92 | >3.7 |

### Conclusions:

In the cell model infected by influenza virus, theaflavins 60% and theaflavins 80% all presented significant inhibitory activity of CPE induced by influenza virus H1N1 (A/PR/8/34), and also presented a good relationship of dose-dependent. The anti-influenza virus activity of theaflavins 80% is more potent than one of theaflavins 60%.

### Example V

### Screening Report

### Evaluation of Inhibitory Activity of Neuraminidase in Influenza Virus (Study of Theaflavins as Anti Influenza Virus (H1N1) Agents)

Samples: Theaflavins 60% (TF60%) and theaflavins 80% (TF80%)

Sample Provider: Jiangsu Dehe Biotechnology Co., Ltd.

### Evaluation of Neuraminidase (NA) Inhibitory Activity

In an enzymatic reaction system, a certain concentration of testing compound and neuraminidase from influenza virus are suspended in buffer solution of the reaction (pH 6.5), then the fluorogenic substrate of MUNANA is added to initiate the reaction, it is incubated for 60 min at 37° C., and the stopper solution is added to terminate the reaction. Under the conditions of excitation wavelength 365 nm and emission wavelength 455 nm, the fluorescence intensity is measured. Fluorescence intensity of the reaction system reflects the enzymatic activity. According to the decreased amount of fluorescence intensity, the inhibitory rate of testing compound against NA activity can be calculated.

### Evaluation of Inhibitory Activity of Cell Pathogy Effect (CPE) Induced by Influenza Virus

Cell: Madin-Darby canine kidney (MDCK). The culture is DMEM culture with 10% serum (Purchased from Gibco). The maintaining culture is non-serum DMEM culture with 5 μg/ml trysogen, and is used for maintaining culture after virus infection.

MDCK cell in logarithmic phase is added to DMEM culture that contains 10% serum, 1×10/ml of MDCK cell mixture is added into the culturing plate with 96 holes, and incubated in 5% CO₂ incubator for 24 hours at temperature of 37° C., the single cell layer is formed. The samples of TF80% and TF60% are diluted by DMEM culture, respectively, to different concentrations for later use. In the experiment, the following groups are designated: experimental group, regular control group and virus infected control group. The subtype of H1N1 influenza virus A/PR/8/34 (200TCIDDD₅₀) is added to MDCK cell, the virus is absorbed for 1 hour at 33° C., then it is taken out, and virus is removed and then added to experimental cultures of different concentrations. They are incubated for 24 hours at temperature of 33° C. and 5% CO₂. Taking out and adding 1% staining solution of crystal violet ammonium oxalate to stain cells for 10 min, washing 96 holes culture plate using water, then drying. 100 μl of 1% SDS is added to each one of holes, vibrate well, and measuring the cellular activity at 570 nm wavelength.

The results of NA inhibitory activity of theaflavins are shown in Table 6. The results of virus (H1N1) inhibitory activity of theaflavins at cellular level are shown in Table 7.

TABLE 6
NA (H1N1) Inhibitory Activity of Theaflavins

| | Concentration | Inhibitory | |
|---|---|---|---|
| Sample | (μg/ml) | Rate % | IC50 (μg/ml) |
| TF60% | 40 | 64.24 ± 1.32 | 28.88 ± 2.32 |
| TF80% | 40 | 81.20 ± 2.53 | 23.63 ± 2.09 |
| Zanzmivir | 0.004 | 93.75 | 2.40 × 10⁻⁴ |

TABLE 7
Virus (H1N1) Inhibitory Activity of Theaflavins at Cellular Level

| Sample | MNCC (μg/ml) | CC50 (μg/ml) | IC50 (μg/ml) | CC50/IC50 |
|---|---|---|---|---|
| TF60% | 25 | >100 | 34.43 ± 11.57 | >2.9 |
| TF80% | 25 | >100 | 27.39 ± 9.92 | >3.7 |

In the screening model of neuraminidase inhibitor, theaflavins 60% and theaflavins 80% all present certain neuraminidase inhibitory activity, their IC₅₀ are 28.88±2.32 μg/ml and 23.63±2.09 μg/ml, respectively. Their NA inhibitory activity are lower than positive control drug. In the cell model infected by influenza virus, theaflavins 60% and theaflavins 80% all presented significant inhibitory activity of CPE induced by influenza virus H1N1 (A/PR/8/34), and also presented a good relationship of dose-dependent. The anti-influenza virus activity of theaflavins 80% is more potent than one of theaflavins 60%.

### Example VI

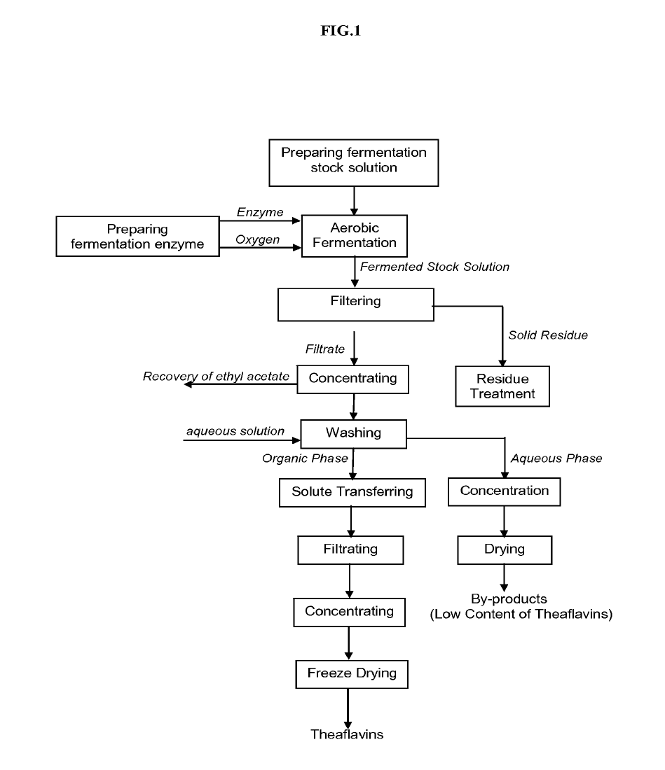

See, FIG. 1. (1) To an example extraction container (2000 ml electric stirring device, flask with three-neck, condensing tube and water bath.), 100 g of large leaf tea is added, followed by the addition of 1000 ml ethyl acetate. The temperature is controlled at a range of 25° C.-60° C. Thus formed mixture is stirred at speed of 30-150 rpm for 30 min. The liquid layer (or extraction solution) is then collected. To the solid residue, another 1000 ml of ethyl acetate is added. The extraction process is repeated. The extracted solution is combined and concentrated at vacuum −0.085 Mpa and 70° C. in a rotating evaporator (Model: RE-200, Manufacturer: Shanghai Kexing Instrument Co.), to a suspension that contains approximate 1% (w/v) of the solid. The suspension is then cooled to ambient temperature and thus formed the fermentation stock solution. (2) The starting enzyme is prepared from 120 g of fresh tea leaves. The fresh tea leaves are sprinkled with pure water or a 0.1% citric acid aqueous solution, then cooled to 0° C. or below and frozen for a minimum of 2 hours. The frozen tea leaves are crushed to small particles of about 80 mesh at a room temperature. Thus prepared enzyme materials are ready to use (storing time is less than 2 hours at ambient temperature). (3) To a fermentation container (glass reactor, Model: DAT serial, Manufacturer: Shanghai Kexing Instrument Co.), 1000 g of fermentation stock solution prepared from step (1) and 100 g of enzyme materials prepared in step (2) are added and oxygen is introduced at flow rate of 600 ml/min, while the mixture is stirred at 30-150 rpm at 20-25° C. Analytical samples are collected from the fermentation container at each 30 min for a total time of 4-8 hours depending on the actual analytical results. When the reaction is complete, both oxygen flow and stirring are stopped. The end point of the reaction is determined by a curve of time-absorption value prepared at 380 nm, e.g. when the absorption value for theaflavins is steady as time increases, the end point is considered to be reached. (4) Immediately after the reaction is stopped, the reaction mixture is filtered through a 120 mesh filter screen. Both the filtrate and the solid residue are collected. The solid residue is treated and then dried as a byproduct. The filtrate was concentrated at temperature of 60-80° C. and vacuum of −0.085 Mpa or above, by using rotating evaporator (Model: RE-200, Manufacturer: Shanghai Kexing Instrument Co.). When the solid content in the solution reached 8% (w/v), the concentrated solution is transferred to a washing container (Pear Shape Separatory Funnel, 500 ml, Manufacturer: Shanghai Shendi Glassware Co.). The concentrated solution (ethyl acetate solution) is washed with 0.1% citric acid aqueous solution. The volume of washing solution is equal to 20% of the concentrated solution, and the ethyl acetate solution is washed 8 times. Both the organic layer and the aqueous layer are collected. The combined aqueous layer is concentrated and dried to yield byproducts. To the organic layer (ethyl acetate layer), 100 ml water is added and the resulted mixture is further concentrated at 60-80° C. till the substantially complete removal of ethyl acetate. The aqueous layer (theaflavins solution) is then collected and cooled to 40° C. The solution is filtered through a 100 mesh filter screen to remove insoluble impurities. The filtrate is collected and concentrated at about 60-80° C. and vacuum of −0.085 Mpa or above to solid content of 40% (w/v). The concentrator used in above steps is a rotating evaporator (Model: RE-200, Manufacturer: shanghai Kexing Instrument Co., Ltd.). The resulted mixture is cooled to −35° C., then lyophilized under a vacuum of 0.6-1 mmHg. Upon the removal of the ice chunk or ice particles and under the same vacuum condition, the temperature of the solid product is increased gradually to 0° C. at a controlled rate at 3° C./hour, then to 25-30° C. at a controlled rate of 5° C./hour. After the solid product is dried under the same vacuum and 25-30° C. for 1-2 hours and the water content reaches 3%-5% in the solid product, the vacuum is released and the solid product is crushed by a micro high speed mill (Model: XA-1, Particle Size: 80-150 mesh, Manufacturer: Jiangyan Yinhe Instrument Co., Ltd.) and screened through a screen grader to give 10.7 g of theaflavins product. Analysis of the product showed the theaflavins content of 40%. (w/w)

### Example VII

A process of manufacturing theaflavins: (1) As shown in FIG. 1, to an extraction container (2000 ml, specially designed and assembled by inventors, which is consisted of electric stirring device, flask with three-neck, condensing tube and water bath.), 100 g of green tea was added, followed by the addition of 1500 ml ethyl acetate. The temperature was controlled at a range of 25° C.-60° C. Thus formed mixture was stirred at speed of 30-150 rpm for 30 min. The organic phase (or extraction solution) was collected. To the solid residue, another 1500 ml of ethyl acetate was added and the above extraction process was repeated. The extracted organic phases were combined and concentrated at vacuum −0.085 Mpa and 70° C. in a rotating evaporator (Model: RE-200, Manufacturer: Shanghai Kexing Instrument Co., Ltd.) to a suspension that contains approximately 0.8% (w/v) of solid. The suspension was then cooled to ambient temperature and thus formed the fermentation stock solution. (2) The starting fermentation enzyme was prepared from 180 g of fresh tea leaves. The fresh tea leaves were sprinkled with pure water or a citric acid aqueous solution (0.5%), then cooled to 0° C. or below and frozen for a minimum of 2 hours. The frozen tea leaves were crashed to small particles of 80 mesh below room temperature. Thus prepared fermentation enzyme was ready to use (storing time is less than 2 hours at ambient temperature). (3) To a fermentation container (glass reactor, Model: DAT serial, Manufacturer: Shanghai Kexing Instrument Co., Ltd), 1500 g of fermentation stock solution prepared from step (1) and 150 g of fermentation enzyme materials prepared in step (2) were added and oxygen was introduced at flow rate of 600 ml/min while the mixture was stirred at 30-150 rpm at 20-25° C. Analytical samples were collected from the fermentation container every 30 min for a total time of 4-8 hours depending on the actual analytical results. Both oxygen flow and stirring were stopped, and thus the process was stopped completely. The end point of the reaction is determined by a curve of time-absorption value prepared at 380 nm, e.g. when the absorption value for theaflavins is steady as time increases, the end point is considered to be reached. (4) Immediately after the reaction was stopped, the reaction mixture was filtered through a 120 mesh filter screen. Both the filtrate and the solid residue were collected. The solid residue was treated and then dried as a byproduct. The filtrate was concentrated at temperature of 60-80° C. and vacuum of −0.085 Mpa or above, by using rotating evaporator (Model: RE-200, Manufacturer: Shanghai Kexing Instrument Co., Ltd.). When the solid content in the solution reached 8% (w/v), the concentrated solution was transferred to a washing container (Pear Shape Separatory Funnel, 500 ml, Manufacturer: Shanghai Shendi Glassware Co., Ltd.). The concentrated solution (ethyl acetate solution) was washed with 0.05% citric acid aqueous solution. The volume of washing solution is equal to 10% of the concentrated solution and the ethyl acetate solution was washed 8 times. Both the organic layer and the aqueous layer were collected. The combined aqueous layer was concentrated and dried to yield byproducts. To the organic layer (ethyl acetate layer), 140 ml of water was added and the resulting mixture was further concentrated at 80° C. or below till the complete removal of ethyl acetate. The aqueous layer (theaflavin solution) was then collected and cooled to 40° C. The solution was filtered through a 100 mesh filter screen to remove insoluble impurities. The filtrate was collected and concentrated at about 60-80° C. and vacuum of −0.085 Mpa or above to solid content of 40% (w/v). The concentrator used in above steps is a rotating evaporator (Model: RE-200, Manufacturer: shanghai Kexing Instrument Co., Ltd.). The resulted mixture was cooled to −35° C., then lyophilized under a vacuum of 0.6-1 mmHg. Upon the removal of the ice chunk or ice particles and under the same vacuum condition, the temperature of the solid product was increased gradually to 0° C. at a controlled rate at 3° C./hour, then to 25-30° C. at a controlled rate of 5° C./hour. After the solid product was dried under the same vacuum at 25-30° C. for 1-2 hours and the water content reached 3%-5% in the solid product, the vacuum was removed. The solid product was crashed by a micro high speed mill (Model: XA-1, Particle Size: 80-150 mesh, Manufacturer: Jiangyan Yinhe Instrument Co., Ltd.) and screened through a screen grader to give 6.4 g of theaflavin product. Analysis of the product showed a theaflavin content of 45.8% (w/w).

### Example VIII

A process of manufacturing theaflavins: (1) As shown in FIG. 1, to an extraction container (5000 ml, specially designed and assembled by inventors, which is consisted of electric stirring device, flask with three-neck, condensing tube and water bath.), 100 g of green tea was added, followed by the addition of 3000 ml ethyl acetate. The temperature was controlled at a range of 25° C.-60° C. Thus formed mixture was stirred and extracted at speed of 30-150 rpm for 30 min. The organic phase (or extraction solution) was collected. To the solid residue, another 3000 ml of ethyl acetate was added and the above extraction process was repeated. The extracted organic phases were combined and concentrated at vacuum −0.085 Mpa and 70° C. in a rotating evaporator (Model: RE-200, Manufacturer: Shanghai Kexing Instrument Co., Ltd.), to a suspension that contains approximately 0.5% of solid (w/v). The suspension was then cooled to ambient temperature and thus formed the fermentation stock solution. (2) The fermentation enzyme was prepared from 180 g of fresh tea leaves. The fresh tea leaves were sprinkled with pure water or a citric acid aqueous solution (1%), then cooled to 0° C. or below and frozen for a minimum of 2 hours. The frozen tea leaves were crashed to small particle size of 80 mesh below room temperature. Thus prepared fermentation enzyme was ready to use (storing time is less than 2 hours at ambient temperature). (3) To a fermentation container (glass reactor, Model: DAT serial, Manufacturer: Shanghai Kexing Instrument Co., Ltd), 3000 g of fermentation stock solution prepared from step (1) and 150 g of enzyme materials prepared in step (2) were added and oxygen was introduced at flow rate of 600 ml/min, while the mixture was stirred at 30-150 rpm at 20° C. Analytical samples were collected from the fermentation container every hour till the fermentation process was essentially complete. The total period of time of fermentation is 4-8 hours depending on the actual test results. Both oxygen flow and stirring were stopped, and thus the process was stopped completely. The end point of the reaction is determined by a curve of time-absorption prepared at 380 nm, e.g. when the absorption value is steady as time increases, the end point is considered to be reached. (4) Immediately after the reaction was stopped, the reaction mixture was filtered through a 120 mesh filter screen. Both the filtrate and the solid residue were collected. The filtrate was concentrated at temperature of 60-80° C. and vacuum of −0.085 Mpa or above, by using rotating evaporator (Model: RE-200, Manufacturer: Shanghai Kexing Instrument Co., Ltd.). When the solid content in the solution reached 15% (w/v), the concentrated solution was transferred to a washing container (Pear Shape Separatory Funnel, 500 ml, Manufacturer: Shanghai Shendi Glassware Co., Ltd.). The concentrated solution (ethyl acetate solution) was washed with 1% citric acid aqueous solution. The ethyl acetate solution was washed 8 times. Both the organic layer and the aqueous layer were collected. The combined aqueous layer was concentrated and dried to yield byproducts. To the organic layer (ethyl acetate layer), 50 ml water was added and the resulted mixture was further concentrated at 80° C. till the complete removal of ethyl acetate. The aqueous layer (theaflavins solution) was then collected and cooled to 30° C. The solution was filtered through a 100 mesh filter screen to remove insoluble impurities. The filtrate was collected and concentrated at about 70° C. and vacuum of −0.085 Mpa or above in a rotating evaporator (Model: RE-200, Manufacturer: shanghai Kexing Instrument Co., Ltd.) to solid content of 25% (w/v). The resulted mixture was cooled to −35° C., then lyophilized under a vacuum of 0.6-1 mmHg. Upon the removal of the ice chunk or ice particles and under the same vacuum condition, the temperature of the solid product was increased gradually to 0° C. at a controlled rate at 3° C./hour, then to 25-30° C. at a controlled rate of 5° C./hour. After the solid product was dried under the same vacuum at 25-30° C. for 1-2 hours and the water content reached 3%-5% in the solid product, the vacuum was removed and the solid product was crashed by a micro high speed mill (Model: XA-1, Particle Size: 80-150 mesh, Manufacturer: Jiangyan Yinhe Instrument Co., Ltd.) and screened through a screen grader to give 6.3 g of theaflavins product. Analysis of the product showed a theaflavins content of 46.1% (w/w).

### Example IX

A process of manufacturing theaflavins: (1) As shown in FIG. 1, to an extraction container (2000 ml, specially designed and assembled by inventors, which is consisted of electric stirring device, flask with three-neck, condensing tube and water bath.), 100 g of green tea was added, followed by the addition of 1000 ml ethyl acetate. The temperature was controlled at a range of 25-60° C. Thus formed mixture was stirred and extracted at speed of 100-150 rpm for 30 min. The liquid layer (or extraction solution) was collected. To the solid residue, another 1000 ml of ethyl acetate was added and the above extraction process was repeated. The extracted solution was combined and concentrated at 60-80° C. and vacuum of −0.085 Mpa or above in a rotating evaporator (Model: RE-200, Manufacturer: Shanghai Kexing Instrument Co., Ltd.), to a suspension that contains approximate 1% of solid (w/v). The suspension was then cooled to ambient temperature and thus formed the fermentation stock solution. (2) The fermentation enzyme was prepared from 220 g of fresh tea leaves. The fresh tea leaves were sprinkled with pure water or a citric acid aqueous solution (0.1%), then cooled to 0° C. or below and frozen for a minimum of 2 hours. The frozen tea leaves were crashed to small particle size of 80 mesh below room temperature. Thus prepared fermentation enzyme was ready to use (storing time is less than 2 hours at ambient temperature). (3) To a fermentation container (glass reactor, Model: DAT serial, Manufacturer: Shanghai Kexing Instrument Co., Ltd), 1000 g of fermentation stock solution prepared from step (1) and 200 g of fermentation enzyme prepared in step (2) was added and oxygen was introduced at flow rate of 600 ml/min, while the mixture was stirred at 30-150 rpm at 20° C. Analytical samples were collected from the fermentation container every hour till the fermentation process was essentially complete. The total period of time of fermentation is 4-8 hours depending on the actual analytical results. Both oxygen flow and stirring were stopped, and thus the process was stopped completely. The end point of the reaction is determined by a curve of time-absorption prepared at 380 nm, e.g. when the absorption value is steady as time increases, the end point is considered to be reached. (4) Immediately after the reaction was stopped, the reaction mixture was filtered through a filter screen. Both the filtrate and the solid residue were collected. The filtrate was concentrated at temperature of 60-80° C. and vacuum of −0.085 Mpa or above, by using rotating evaporator (Model: RE-200, Manufacturer: Shanghai Kexing Instrument Co., Ltd.). When the solid content in the solution reached 8% (w/v), the concentrated solution was transferred to a washing container (Pear Shape Separatory Funnel, 500 ml, Manufacturer: Shanghai Shendi Glassware Co., Ltd.). The concentrated solution (ethyl acetate solution) was washed with 0.1% citric acid aqueous solution. The volume of washing solution is equal to 20% of the concentrated solution, and the ethyl acetate solution was washed 8 times. Both the organic layer and the aqueous layer were collected. The combined aqueous layer was concentrated and dried to yield byproducts. To the organic layer (ethyl acetate layer), 100 ml water was added and the resulted mixture was further concentrated at 60-80° C. till the complete removal of ethyl acetate. The aqueous layer (theaflavins solution) was then collected and cooled to 60° C. The solution was filtered through a 100 mesh filter screen to remove insoluble impurities. The filtrate was collected and concentrated at about 70° C. and vacuum of −0.085 Mpa or above to solid content of 80% (w/v). The concentrator used in above steps is a rotating evaporator (Model: RE-200, Manufacturer: shanghai Kexing Instrument Co., Ltd.) The resulting mixture was cooled to −35° C., then lyophilized under a vacuum of 0.6-1 mmHg. Upon the removal of the ice chunk or ice particles and under the same vacuum condition, the temperature of the solid product was increased gradually to 0° C. at a controlled rate at 3° C./hour, then to 25-30° C. at a controlled rate of 5° C./hour. After the solid product was dried under the same vacuum at 25-30° C. for 1-2 hours and the water content reached 3%-5% in the solid product, the vacuum was removed and the solid product was crashed by a micro high speed mill (Model: XA-1, Particle Size: 80-150 mesh, Manufacturer: Jiangyan Yinhe Instrument Co., Ltd.) and screened through a screen grader to give 6.2 g of theaflavins product. Analysis of the product showed a theaflavins content of 47.8% (w/w).

### Example X

A process of manufacturing theaflavins: (1) As shown in FIG. 1, to an extraction container (3000 ml, specially designed and assembled by inventors, which is consisted of electric stirring device, flask with three-neck, condensing tube and water bath.), 200 g of green tea was added, followed by the addition of 2000 ml ethyl acetate. The temperature was controlled at a range of 25° C.-60° C. Thus formed mixture was stirred and extracted at speed of 30-150 rpm for 30 min. The liquid layer (or extraction solution) was collected. To the solid residue, another 2000 ml of ethyl acetate was added and the above extraction process was repeated once. The extracted solution was combined and concentrated at vacuum −0.085 Mpa and 70° C. in a rotating evaporator (Model: RE-200, Manufacturer: Shanghai Kexing Instrument Co., Ltd.), to a suspension that contains approximate 1% of solid (w/v). The suspension was then cooled to ambient temperature and thus formed the fermentation stock solution. (2) The fermentation enzyme was prepared from 220 g of fresh tea leaves. The fresh tea leaves were sprinkled with pure water or a citric acid aqueous solution (0.1%), then cooled to 0° C. or below and frozen for a minimum of 2 hours. The frozen tea leaves were crashed to small particle size of 80 mesh below room temperature. Thus prepared fermentation enzyme was ready to use (storing time is less than 2 hours at ambient temperature). (3) To a fermentation container (glass reactor, Model: DAT serial, Manufacturer: Shanghai Kexing Instrument Co., Ltd), 2000 g of fermentation stock solution prepared from step (1) and 200 g of fermentation enzyme prepared in step (2) was added and oxygen was introduced at flow rate of 600 ml/min, while the mixture was stirred at 30-150 rpm at 25° C. Analytical samples were collected from the fermentation container every hour till the fermentation process was essentially complete. Both oxygen flow and stirring were stopped, and thus the process was stopped completely. The total period of time of fermentation is 4-8 hours depending on the actual test results. The end point of the reaction is determined by a curve of time-absorption prepared at 380 nm, e.g. when the absorption value is steady as time increases, the end point is considered to be reached. (4) Immediately after the reaction was stopped, the reaction mixture was filtered through a 120 mesh filter screen. Both the filtrate and the solid residue were collected. The filtrate was concentrated at temperature of 60-80° C. and vacuum of −0.085 Mpa or above, by using rotating evaporator (Model: RE-200, Manufacturer: Shanghai Kexing Instrument Co., Ltd.). When the solid content in the solution reached 8% (w/v), the concentrated solution was transferred to a washing container (Pear Shape Separatory Funnel, 500 ml, Manufacturer: Shanghai Shendi Glassware Co., Ltd.). The concentrated solution (ethyl acetate solution) was washed with 0.1% citric acid aqueous solution. The volume of washing solution is equal to 20% of the concentrated solution, and the ethyl acetate solution was washed 20 times. Both the organic layer and the aqueous layer were collected. The combined aqueous layer was concentrated to yield byproducts. To the organic layer (ethyl acetate layer), 100 ml water was added and the resulted mixture was further concentrated at 60-80° C. till the complete removal of ethyl acetate. The aqueous layer (theaflavins solution) was then collected and cooled to 40° C. The solution was filtered through a 100 mesh filter screen to remove insoluble impurities. The filtrate was collected and concentrated at about 60-80° C. and vacuum of −0.085 Mpa or above in a rotating evaporator (Model: RE-200, Manufacturer: shanghai Kexing Instrument Co., Ltd.) to solid content of 40% (w/v). The resulted mixture was cooled to −35° C., and then lyophilized under a vacuum of 0.6-1 mmHg. Upon the removal of the ice chunk or ice particles and under the same vacuum condition, the temperature of the solid product was increased gradually to 0° C. at a controlled rate at 3° C./hour, then to 25-30° C. at a controlled rate of 5° C./hour. After the solid product was dried under the same vacuum and 25-30° C. for 1-2 hours and the water content reached 3%-5% in the solid product, the vacuum was removed and the solid product was crashed by a micro high speed mill (Model: XA-I, Particle Size: 80-150 mesh, Manufacturer: Jiangyan Yinhe Instrument Co., Ltd.) and screened through a screen grader to give 13.12 g of theaflavin product. Analysis of the product showed a theaflavins content of 47% (w/w).

### Example XI

A process of manufacturing theaflavins: (1) As shown in FIG. 1, to an extraction container (2000 ml, specially designed and assembled by inventors, which is consisted of electric stirring device, flask with three-neck, condensing tube and water bath.), 100 g of green tea was added, followed by the addition of 1000 ml ethyl acetate. The temperature was controlled at a range of 25-60° C. Thus formed mixture was stirred and extracted at speed of 30-150 rpm for 30 min. The liquid layer (or extraction solution) was collected. To the solid residue, another 1000 ml of ethyl acetate was added and the above extraction process was repeated once. The extracted solution was combined and concentrated at vacuum −0.085 Mpa or at 70° C. in a rotating evaporator (Model: RE-200, Manufacturer: Shanghai Kexing Instrument Co., Ltd.), to a suspension that contains approximate 1% of solid (w/v). The suspension was then cooled to ambient temperature and thus formed a fermentation stock solution. (2) The fermentation enzyme was prepared from 220 g of fresh tea leaves. The fresh tea leaves were sprinkled with pure water or a citric acid aqueous solution (0.1%), then cooled to 0° C. or below and frozen for a minimum of 2 hours. The frozen tea leaves were crashed to small particle size of 80 mesh below room temperature. Thus prepared fermentation enzyme was ready to use (storing time is not more than 2 hours at ambient temperature). (3) To a fermentation container (glass reactor, Model: DAT serial, Manufacturer: Shanghai Kexing Instrument Co., Ltd), 1000 g of fermentation stock solution prepared from step (1) and 200 g of fermentation enzyme prepared in step (2) was added and oxygen was introduced at flow rate of 600 ml/min, while the mixture was stirred at 30-150 rpm at 25° C. Analytical samples were collected from the fermentation container every hour till the fermentation process was essentially complete. Both oxygen flow and stirring were stopped, and thus the process was stopped completely. The total period of time of fermentation is 4-8 hours depending on the actual test results. The end point of the reaction is determined by a curve of time-absorption prepared at 380 nm, e.g. when the absorption value is steady as time increases, the reaction is considered to be completed. (4) Immediately after the reaction was stopped, the reaction mixture was filtered through a 120 mesh filter screen. Both the filtrate and the solid residue were collected. The filtrate was concentrated at temperature of 60-80° C. and vacuum of −0.085 Mpa or above, by using rotating evaporator (Model: RE-200, Manufacturer: Shanghai Kexing Instrument Co., Ltd.). When the solid content in the solution reached 8% (w/v), the concentrated solution was transferred to a washing container (Pear Shape Separatory Funnel, 500 ml, Manufacturer: Shanghai Shendi Glassware Co., Ltd.). The concentrated solution (ethyl acetate solution) was washed with 0.1% citric acid aqueous solution. The volume of washing solution is equal to 20% of the concentrated solution and the ethyl acetate solution was washed once. Both the organic layer and the aqueous layer were collected. The combined aqueous layer was concentrated and dried to yield byproducts. To the organic layer (ethyl acetate layer), 100 ml water was added and the resulted mixture was further concentrated at 60-80° C. till the complete removal of ethyl acetate. The aqueous layer (theaflavins solution) was then collected and cooled to 60° C. The solution was filtered through a 100 mesh filter screen to remove insoluble impurities. The filtrate was collected and concentrated at about 70° C. and vacuum of −0.085 Mpa or above in a rotating evaporator (Model: RE-200, Manufacturer: shanghai Kexing Instrument Co., Ltd.) to solid content of 50% (w/v). The resulted mixture was cooled to −35° C., then lyophilized under a vacuum of 0.6-1 mmHg. Upon the removal of the ice chunk or ice particles and under the same vacuum condition, the temperature of the solid product was increased gradually to 0° C. at a controlled rate at 3° C./hour, then to 25-30° C. at a controlled rate of 5° C./hour. After the solid product was dried under the same vacuum and 25-30° C. for 1-2 hours and the water content reached 3%-5% in the solid product, the vacuum was removed and the solid product was crashed by a micro high speed mill (Model: XA-I, Particle Size: 80-150 mesh, Manufacturer: Jiangyan Yinhe Instrument Co., Ltd.) and screened through a screen grader to give 6.1 g of theaflavins product. Analysis of the product showed a theaflavins content of 46% (w/w).

### Example XII

(1) To a extraction container (2000 ml, specially designed and assembled by inventors, which is consisted of electric stirring device, flask with three-neck, condensing tube and water bath.), 100 g of green tea was added, followed by the addition of 1000 ml ethyl acetate. The temperature was controlled at a range of 25° C.-60° C. Thus formed mixture was stirred at speed of 30-150 rpm for 30 min. The liquid layer (or extraction solution) was then collected. To the solid residue, another 1000 ml of ethyl acetate was added and the above extraction process was repeated. The extracted solution was combined and concentrated at vacuum −0.085 Mpa and 70° C. in a rotating evaporator (Model: RE-200, Manufacturer: Shanghai Kexing Instrument Co., Ltd.), to a suspension that contains approximate 1% (w/v) of the solid. The suspension was then cooled to ambient temperature and thus formed the fermentation stock solution. (2) The starting enzyme was prepared from 120 g of fresh tea leaves. The fresh tea leaves were sprinkled with pure water or a 0.1% citric acid aqueous solution, then cooled to 0° C. or below and frozen for a minimum of 2 hours. The frozen tea leaves were crushed to small particles of about 80 mesh at a room temperature. Thus prepared enzyme materials were ready to use (storing time is not more than 2 hours at ambient temperature). (3) To a fermentation container (glass reactor, Model: DAT serial, Manufacturer: Shanghai Kexing Instrument Co., Ltd), 1000 g of fermentation stock solution prepared from step (1) and 100 g of enzyme materials prepared in step (2) were added and oxygen was introduced at flow rate of 600 ml/min, while the mixture was stirred at 30-150 rpm at 20° C. Analytical samples were collected from the fermentation container at each 30 min for a total time of 4-8 hours depending on the actual analytical results. When the reaction was complete, both oxygen flow and stirring were stopped. The end point of the reaction is determined by a curve of time-absorption value prepared at 380 nm, e.g. when the absorption value for theaflavins is steady as time increases, the end point is considered to be reached. (4) Immediately after the reaction was stopped, the reaction mixture was filtered through a 120 mesh filter screen. Both the filtrate and the solid residue were collected. The solid residue was treated and then dried as a byproduct. The filtrate was concentrated at temperature of 60-80° C. and vacuum of −0.085 Mpa or above, by using rotating evaporator (Model: RE-200, Manufacturer: Shanghai Kexing Instrument Co., Ltd.). When the solid content in the solution reached 8% (w/v), the concentrated solution was transferred to a washing container (Pear Shape Separatory Funnel, 500 ml, Manufacturer: Shanghai Shendi Glassware Co., Ltd.). The concentrated solution (ethyl acetate solution) was washed with 0.1% citric acid aqueous solution. The volume of washing solution is equal to 20% of the concentrated solution, and the ethyl acetate solution was washed 8 times. Both the organic layer and the aqueous layer were collected. The combined aqueous layer was concentrated and dried to yield byproducts. To the organic layer (ethyl acetate layer), 100 ml water was added and the resulted mixture was further concentrated at 60-80° C. till the substantially complete removal of ethyl acetate. The aqueous layer (theaflavins solution) was then collected and cooled to 40° C. The solution was filtered through a 100 mesh filter screen to remove insoluble impurities. The solution was filtered through a 100 mesh filter screen to remove insoluble impurities. The filtrate was collected and concentrated at about 60-80° C. and vacuum of −0.085 Mpa or above to solid content of 20-60% (w/v). The concentrator used in above steps is a rotating evaporator (Model: RE-200, Manufacturer: shanghai Kexing Instrument Co., Ltd.) The concentrate was introduced to spray dryer (Model YC-015, Manufacturer: Wuxi Dongsheng Spray-granulating & Srying Equipment Plant) by pump. Inlet air temperature of the spray dryer was controlled at 180-220° C., when outlet air temperature reached at 80-100° C., collected the dried product, and then screened through a screen grader to give 6.6 g of theaflavins product. Analysis of the product showed the theaflavins content of 45.8%. (w/w).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described compositions and modes for carrying out the invention which are obvious to those skilled in the art or related fields are intended to be within the scope of the following claims.

1. A process for producing a purified extract comprising between about 40% and about 90% theaflavins, the process comprising:
producing tea leaves;
combining an organic solvent with the tea leaves and extracting polyphenols from the tea leaves to produce an organic stock substrate solution;
producing a second batch of tea leaves;
grinding the second batch of tea leaves to produce stock fermentation enzyme; combining the stock substrate solution with the stock fermentation enzyme to produce a fermentation mixture;
fermentation of the mixture to produce theaflavins; and,
separating the theaflavins from the fermentation mixture to produce purified theaflavin extract.

2. The process according to claim 1 wherein the tea leaves are Camellia sinensis var. assamica and organic solvent is selected from the group consisting of at least one ester [e.g. ethyl acetate], alcohol, ketone, and ether.

3. A purified theaflavin extract produced by the process of claim 1 comprising between about 50% and about 85% theaflavins.

4. A purified theaflavin extract according to claim 3 comprising between about 60% and about 80% theaflavins.

5. A purified extract according to claim 4 comprising less than about 5% TF1, between about 10% and about 60% TF2a, between about 5% and about 35% TF2b, and between about 10% and about 65% TF3.

6. A purified extract according to claim 5 comprising less than about 4% TF1, between about 20 and about 50% TF2a, between about 5 and about 20% TF2b, and between about 20 and about 50% TF3.

7. A purified extract according to claim 6 comprising about 80% theaflavins wherein the extract comprises less than about 3% TF1, about 30% TF2a, about 10% TF2b, and about 40% TF3.

8. A individual dosage composition for the control of a physiological disorder comprising about 5% to about 95% theaflavins in a pharmaceutically acceptable vehicle or a dietary supplement vehicle.

9. The individual dosage composition according to claim 8 comprising an effective amount of substantially only one theaflavin species selected from the group consisting of TF1, TF2a, TF2b, and TF3.

10. The individual dosage composition according to claim 8 comprising an effective amount of substantially only two theaflavin species selected from the group consisting of TF1 and TF2a, TF1 and TF2b, TF1 and TF3, TF2a and TF2b, TF2a and TF3, TF2b and TF3.

11. The individual dosage composition according to claim 8 comprising an effective amount of substantially only three theaflavin species selected from the group consisting of TF1 and TF2a and TF2b, TF3 and TF2a and TF2b, TF3 and TF1 and TF2b; and, TF3 and TF1 and TF2a.

12. The individual dosage composition according to claim 8 comprising an effective amount of substantially four theaflavin species TF1, TF3, TF2a, and TF2b.

13. An oral dosage form comprising an effective amount of a purified extract according to claim 5.

14. An oral dosage form comprising an effective amount of a composition according to claim 9.

15. An oral dosage form comprising an effective amount of a composition according to claim 10.

16. An oral dosage form comprising an effective amount of a composition according to claim 11.

17. A method of treatment of a human physiological disorder comprising administering an oral dosage form according to claim 13.

18. A method of treatment of a human physiological disorder comprising administering an oral dosage form according to claim 14.

19. A method of treatment of a human physiological disorder comprising administering an oral dosage form according to claim 15.

20. A method of treatment of a human physiological disorder comprising administering an oral dosage form according to claim 16.

21. A method for treatment of a mammal infected with influenza virus comprising administering an anti-influenza effective amount of a purified extract comprising between about 40% and about 90% theaflavins, wherein the extract is obtained by a process comprising:
providing tea leaves;
combining an organic solvent with the tea leaves and extracting polyphenols from the tea leaves to produce an organic stock substrate solution;
providing a second batch of tea leaves;
grinding the second batch of tea leaves to produce stock fermentation enzyme;
combining the stock substrate solution with the stock fermentation enzyme to produce a fermentation mixture;
fermentation of the mixture to produce theaflavins; and,
separating the theaflavins from the fermentation mixture to produce purified theaflavin extract.

22. The method of claim 21 wherein the tea leaves are Camellia sinensis var. assamica and organic solvent is selected from the group consisting of at least one ester, alcohol, ketone, ether and combinations thereof.

23. The method of claim 21 wherein the theaflavin extract comprises between about 50% and about 85% theaflavins.

24. The method of claim 21 wherein the theaflavin extract comprises between about 60% and about 80% theaflavins.

25. The method of claim 21 wherein the theaflavin extract comprises less than about 5% TF1, between about 10% and about 60% TF2a, between about 5% and about 35% TF2b, and between about 10% and about 65% TF3.

26. The method of claim 21 wherein the theaflavin extract comprises less than about 4% TF1, between about 20 and about 50% TF2a, between about 5 and about 20% TF2b, and between about 20 and about 50% TF3.

27. The method of claim 21 wherein the theaflavin extract comprises less than about 3% TF1, about 30% TF2a, about 10% TF2b, and about 40% TF3.

28. The method of claim 21 wherein the theaflavin extract is administered in the form of an oral dosage form.
